(19) United States
(12) Patent Application Publication
(10) Pub. No.: US 2021/0178289 A1
(43) Pub. Date: Jun. 17, 2021

(54) METHODS OF ASSAYING TROPOLONE

(71) Applicant: LONZA LTD, Visp (CH)

(72) Inventor: Benjamin Alexander WILKES, Visp (CH)

(21) Appl. No.: 16/769,842

(22) PCT Filed: Dec. 4, 2018
(86) PCT No.: PCT/US2018/063822

§ 371 Date: Jun. 4, 2020

Related U.S. Application Data
(60) Provisional application No. 62/594,863, filed on Dec. 5, 2017.

(51) Int. Cl. B01D 15/08; C07K 16/00; C07C 49/717

(52) U.S. Cl. CPC B01D 15/08; C07C 49/717; C07K 16/00

(57) ABSTRACT

Disclosed herein are methods useful for detecting and/or quantifying tropolone in samples, e.g., generated during the production of and in final formulations of a product, e.g., a recombinant protein, e.g., an antibody. Tropolone and derivatives thereof (cycloheptatriene ketones) are separated from a mixture by adding a partially or fully fluorinated alkyl or aryl (e.g. pentafluorophenyl-propyl), which (covalently) binds the tropolone-like compound. Then it is assayed via UV or tandem mass spectrometry. A reaction mixture of tropolone-like compounds and fluorinated alkyl or aryl is also disclosed.

This application claims priority to U.S. Application 62/594,863 filed Dec. 5, 2017, the entire contents of which is incorporated herein by reference.

### FIELD OF THE INVENTION

The present disclosure relates to methods of detecting and/or quantifying tropolone during the production of a product, e.g., a recombinant protein, e.g., an antibody.

### BACKGROUND

Tropolone (2-hydroxy-2,4,6-cycloheptatrien-1-one) is a small molecule used in cell culture media to facilitate uptake of metal ions, essential for growth of cells such as those used in biomanufacturing. Because tropolone is a synthetic chemical added to cell culture during the manufacturing process of products, regulatory agencies governing biological products often require that tropolone clearance be demonstrated.

Therefore, a need exists for methods of separating, detecting, and quantifying tropolone in a variety of biopharmaceutical products in a simple, rapid, efficient manner.

### SUMMARY

Methods and compositions described herein provide for quickly and easily separating a compound of Formula I, e.g., tropolone, from other sample components and testing for a compound of Formula I, e.g., tropolone, levels and clearance. This allows evaluation of product purity. Methods and compositions described herein can minimize regulatory delay and time and resource expenditure testing for compounds of Formula I, e.g., tropolone.

Accordingly, in one aspect the invention is directed to a method of separating a compound of Formula I, e.g., tropolone, from another component of a sample comprising:

contacting the sample with a partially or fully fluorinated alkyl or aryl, e.g., a fluorophenyl, e.g., a pentafluorophenylpropyl, moiety, under conditions wherein the compound of Formula I, e.g., tropolone, associates with, e.g., binds to or is retained by, the moiety to a greater extent than the component,

thereby separating the compound of Formula I, e.g., tropolone, from the component, wherein Formula I is:

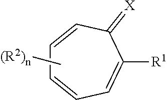

and wherein:

X is O or S;

R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;

each R² is independently C₁-C₆ alkyl, C₁-C₆ heteroalkyl, N(R^{4a})(R^{4b}), C(O)N(R^{4a})(R^{4b}), or N(R^{4a})C(O)R5; or

two R² are joined to form a heterocyclyl ring optionally substituted with one or more R⁶; or R¹ and R² are joined to form a heterocyclyl ring optionally substituted with one or more R⁶;

R³ is hydrogen, C₁-C₆ alkyl, or C₁-C₆ heteroalkyl;

R^{4a} and R^{4b} are independently hydrogen, C₁-C₆ alkyl, or C₁-C₆ heteroalkyl;

R⁵ is C₁-C₆ alkyl or C₁-C₆ heteroalkyl;

each R⁶ is independently C₁-C₆ alkyl, C₁-C₆ heteroalkyl, halo, oxo, or cyano; and

n is 0, 1, 2, 4, or 5.

In another aspect, the invention is directed to a method of evaluating the presence, e.g., the level, of a compound of Formula I, e.g., tropolone, in a sample comprising a product, comprising:

a) i) providing an aliquot of a sample, e.g., a compound of Formula I (e.g., tropolone) depleted phase, e.g., a mobile phase, wherein the compound of Formula I, e.g., tropolone, has been separated from another component of the sample, or
ii) subjecting the sample to conditions wherein the compound of Formula I, e.g., tropolone, is separated from another component of the sample, e.g., to form a compound of Formula I, e.g., tropolone, enriched phase or aliquot and a compound of Formula I, e.g., tropolone, depleted phase or aliquot; and

b) evaluating the presence, e.g., the level, of the compound of Formula I, e.g., tropolone, e.g., determining a value for the level of the compound of Formula I, e.g., tropolone, in the sample:
i) using tandem mass spectrometry (MS²), or
ii) using ultraviolet (UV) absorption, e.g., UV absorption at about 242 nm or about 238 nm,
   thereby analyzing the sample.

In another aspect, the invention is directed to a reaction mixture comprising a partially or fully fluorinated alkyl or aryl, e.g., a fluorophenyl, e.g., a pentafluorophenylpropyl, moiety, and a sample comprising a compound of Formula I, e.g., tropolone, another component, and optionally a product.

In another aspect, the invention is directed to a method of manufacturing a product, e.g., a recombinant polypeptide, comprising providing a sample comprising the product and optionally a compound of Formula I, e.g., tropolone, wherein:

the sample is analyzed by a method described herein, or

the compound of Formula I, e.g., tropolone, is separated from another component of the sample by a method described herein.

### BRIEF DESCRIPTION OF THE DRAWINGS

FIG. 1 shows two views of a chromatogram of tropolone separation by RP-HPLC using UV detection; the bottom view is an expanded view of the top view.

FIG. 2 shows total ion current (TIC) traces of the SRM transitions of Table 1 after separation using the Luna-NH₂ column.

FIG. 3 shows TIC traces of the SRM transitions of Table 1 after separation using the Discovery HS F5-3 (Supelco) column.

FIG. 4 shows a graph showing the calibration curve plot of tropolone standards in water and measuring linear range.

FIG. 5 shows TIC traces of three in process samples, each showing no tropolone peak.

FIG. 6 shows TIC traces of three in process samples either spiked with tropolone (top three) or not spiked with tropolone (bottom three).

FIG. 7 shows two chromatograms detecting tropolone in a tropolone standard processed via the chromatography method determined in Example 2, using UV absorption at 242 nm (top) and 238 nm (bottom).

FIGS. 8A and 8B show detailed parameters of an exemplary LC method of the disclosure.

### DETAILED DESCRIPTION

For recombinant biopharmaceutical proteins to be acceptable for administration to human patients, it is important that residual contaminants resulting from the manufacture and purification process are removed from the final biological product, e.g., recombinant polypeptide. These process contaminants include compounds added to culture medium in the course of culturing cells and purifying biological products.

U.S. and foreign regulations often require removal of such contaminants. For example, the U.S. Food and Drug Administration (FDA) requires that biopharmaceuticals intended for in vivo human use should be as free as possible of extraneous immunoglobulin and non-immunoglobulin impurities, and requires tests for detection and quantitation of potential impurities. As well, the International Conference on Harmonization (ICH) provides guidelines on test procedures and acceptance criteria for biotechnological/biological products.

Tropolone (2-hydroxy-2,4,6-cycloheptatrien-1-one) is a 7-membered aromatic ring. It has several uses, including as an antioxidant in cosmetics and topical pharmaceutical formulations, as a UV-absorber in sun-screen, and as a catechol-O-methyl-transferase (COMT) inhibitor. Tropolone can be added to cell culture media to facilitate the uptake of metal ions in cultured cells. In some embodiments, tropolone is added to cell culture media at a concentration less than or equal to 0.1, 0.5, 1, 1.25, 1.5, 1.75, 2, 2.25, 2.5, 2.75, 3, 3.5, 4, 4.5, 5, 6, 7, 8, 9, or 10 mg/ml.

In some embodiments, a compound of Formula I, e.g., tropolone, can be added to cell culture media to facilitate the uptake of metal ions in cultured cells. In some embodiments, a compound of Formula I, e.g., tropolone, is added to cell culture media at a concentration less than or equal to 0.1, 0.5, 1, 1.25, 1.5, 1.75, 2, 2.25, 2.5, 2.75, 3, 3.5, 4, 4.5, 5, 6, 7, 8, 9, or 10 mg/ml.

As a synthetic chemical added to a culture of cells used to produce a biological product, many regulatory agencies require demonstration of clearance of compounds of Formula I, e.g., tropolone, from biological products, e.g., before they can be declared safe for in vivo human use. Many methods of manufacturing or producing biological products comprise affinity chromatography steps, e.g., use columns comprising resins that selectively retain the desired biological product, and it is expected that compounds of Formula I, e.g., tropolone, would pass through such affinity columns prior to elution of the desired biological product. Any compound of Formula I, e.g., tropolone, remaining could be assayed in samples of the biological product using: (i) suitable chromatography steps to separate the possible remaining compound of Formula I, e.g., tropolone, from other components of the biological product, and (ii) suitable detection and/or quantification steps to determine the presence and abundance of compound of Formula I, e.g., tropolone. Suitable chromatography steps and detection methods are described herein.

The present disclosure describes, inter alia, methods of analyzing samples comprising a product and optionally a compound of Formula I, e.g., tropolone, to determine a value for the level of compound of Formula I, e.g., tropolone, present in the sample, wherein the method is superior with regard to one or more of linear range, precision, accuracy, and limits of detection when compared to previously available methods (e.g., RP-HPLC and UV/fluorescence detection). In some embodiments, the methods of the disclosure are unaffected or not significantly deleteriously affected (e.g., approximately unaffected) with regard to one or more of linear range, precision, accuracy, and limits of detection over a range of products and/or product formulations when compared to previously available methods (e.g., RP-HPLC and UV/fluorescence detection). For example, a method of the disclosure may determine a value for a level of a compound of Formula I, e.g., tropolone, in samples comprising a variety of buffer components with no significant drop in accuracy, whereas previously available methods may determine a value for a level of a compound of Formula I, e.g., tropolone, in samples comprising one buffer component but exhibit a decrease in accuracy when determining a value for a level of a compound of Formula I, e.g., tropolone, in samples comprising another buffer component.

In some embodiments, methods of the disclosure have a linear range, with regard to determining a value for the level of a compound of Formula I, e.g., tropolone, present in the sample, of between about 0.1-10000, 0.2-8000, 0.3-7000, 0.4-6000, 0.5-5000, 0.5-4000, 0.5-3000, 0.5-2000, or 0.5-1000 μg/ml, e.g., 0.5-1000 μg/ml. In some embodiments, methods of the disclosure have a lower limit of a linear range, with regard to determining a value for the level of a compound of Formula I, e.g., tropolone, present in the sample, of about 0.01, 0.05, 0.1, 0.2, 0.3, 0.35, 0.4, 0.45, 0.5, 0.6, 0.7, 0.8, 0.9, or 1 μg/ml, e.g., 0.5 μg/ml. In some embodiments, methods of the disclosure have an upper limit of a linear range, with regard to determining a value for the level of a compound of Formula I, e.g., tropolone, present in the sample, of about 500, 600, 700, 800, 900, 1000, 1200, 1400, 1600, 1800, 2000, 3000, 4000, 5000, 6000, 7000, 8000, 9000, or 10,000 μg/ml, e.g., 1000 μg/ml.

In some embodiments, methods of the disclosure have a precision, with regard to determining a value for the level of a compound of Formula I, e.g., tropolone, present in the sample, represented by the standard deviation between replicate samples. In the same embodiments, the precision can be less than or equal to about 50, 40, 30, 25, 20, 19, 18, 17, 16, 15, 14, 13, 12, 11, 10, 9, 8, 7, 6, 5, 4, 3, 2, or 1%, e.g., 17, 16.5, or 16%.

In some embodiments, methods of the disclosure have an accuracy, with regard to determining a value for the level of a compound of Formula I, e.g., tropolone, present in the sample, represented by average single point spike recovery in three different samples. In the same embodiments, the accuracy can be greater than or equal to about 70, 75, 80, 81, 82, 83, 84, 85, 86, 87, 88, 89, 90, 91, 92, 93, 94, or 95%, e.g., 91%.

In some embodiments, methods of the disclosure have a lower limit of detection with regard to determining a value for the level of a compound of Formula I, e.g., tropolone, present in the sample. In the same embodiments, the lower limit of detection can be about 1, 1.5, 2, 2.5, 3, 3.5, 4, 4.5, 5, 5.5, 6, 6.5, 7, 7.5, 8, 8.5, 9, 9.5, or 10 μg/ml.

The present disclosure further describes, inter alia, methods of manufacturing a product, e.g., a recombinant polypeptide, wherein samples of the product are analyzed by methods of analyzing samples described herein for the presence or level of a compound of Formula I, e.g., tropolone.

In some embodiments, the sample is a sample of a cosmetic formulation, e.g., comprising a product for use in a cosmetic formulation.

In some embodiments, the sample is a sample of a topical pharmaceutical formulation, e.g., comprising a product for use in a pharmaceutical formulation.

In some embodiments, the sample is a sample of a sun-screen, e.g., comprising a product for use in a sun-screen, e.g., a compound of Formula I, e.g., tropolone, and/or another product for use in a sun-screen, e.g., another UV-blocker.

In some embodiments, the sample is a sample of COMT inhibitor, e.g., comprising a product for use as a COMT inhibitor, e.g., a compound of Formula I, e.g., tropolone, and/or another product for use as a COMT inhibitor. In some embodiments, the sample is a sample comprising L-DOPA (e.g., levodopa or L-3,4-dihydroxyphenylalanine) and/or an aromatic L-amino acid decarboxylase inhibitor (e.g., DOPA decarboxylase inhibitor, DDCI, or AAADI).

The present disclosure further describes, inter alia, reaction mixtures comprising a fluorophenyl moiety, e.g., a pentafluorophenylpropyl group, and a sample, wherein the sample comprises a compound of Formula I, e.g., tropolone, another component, and optionally a product. In an embodiment, such reaction mixtures may be useful for separating a compound of Formula I, e.g., tropolone, from the component and/or from the product, and, in further embodiments, subsequently for detecting the presence of or determining the level of a compound of Formula I, e.g., tropolone. The moieties of the reaction mixture may be associated with, e.g., bound to, e.g., covalently bound to, a substrate, wherein the substrate comprises an insoluble substrate, e.g., a chromatography matrix, resin, gel, or beads, e.g., a silica, agarose, cellulose, dextran, polyacrylamide, or latex matrix, resin, gel, or beads.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “a cell” can mean one cell or more than one cell.

As used herein, “about” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “semi-quantitative” refers to the comparative assessment of different chemical species by mass spectrometry without reference to specific standards for each individual species.

As used herein, the term “endogenous” refers to any material from or naturally produced inside an organism, cell, tissue or system.

As used herein, the term “exogenous” refers to any material introduced to or produced outside of an organism, cell, tissue or system. Accordingly, “exogenous nucleic acid” refers to a nucleic acid that is introduced to or produced outside of an organism, cell, tissue or system. In an embodiment, sequences of the exogenous nucleic acid are not naturally produced, or cannot be naturally found, inside the organism, cell, tissue, or system that the exogenous nucleic acid is introduced into. In one embodiment, the sequences of the exogenous nucleic acids are non-naturally occurring sequences, or encode non-naturally occurring products.

As used herein, the term “heterologous” refers to any material from one species, when introduced to an organism, cell, tissue or system from a different species.

As used herein, the terms “nucleic acid,” “polynucleotide,” or “nucleic acid molecule” are used interchangeably and refers to deoxyribonucleic acid (DNA) or ribonucleic acid (RNA), or a combination of a DNA or RNA thereof, and polymers thereof in either single- or double-stranded form. The term “nucleic acid” includes, but is not limited to, a gene, cDNA, or an mRNA. In one embodiment, the nucleic acid molecule is synthetic (e.g., chemically synthesized or artificial) or recombinant. Unless specifically limited, the term encompasses molecules containing analogues or derivatives of natural nucleotides that have similar binding properties as the reference nucleic acid and are metabolized in a manner similar to naturally or non-naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. In one embodiment, a protein may comprise of more than one, e.g., two, three, four, five, or more, polypeptides, in which each polypeptide is associated to another by either covalent or non-covalent bonds/interactions. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others.

As used herein, “product” refers to a molecule, nucleic acid, polypeptide, or any hybrid thereof, that is produced, e.g., expressed, by a cell which has been modified or engineered to produce the product. In one embodiment, the product is a naturally occurring product or a non-naturally occurring product, e.g., a synthetic product. In one embodiment, a portion of the product is naturally occurring, while another portion of the product is non-naturally occurring. In one embodiment, the product is a polypeptide, e.g., a recombinant polypeptide. In one embodiment, the product is suitable for diagnostic or pre-clinical use. In another embodiment, the product is suitable for therapeutic use, e.g., for treatment of a disease. In one embodiment, the product is selected from Table 1, Table 2, Table 3, or Table 4. In one embodiment, the modified or engineered cells comprise an exogenous nucleic acid that controls expression or encodes the product. In other embodiments, the modified or engineered cells comprise other molecules, e.g., that are not nucleic acids, that controls the expression or construction of the product in the cell.

In one embodiment, the modification of the cell comprises the introduction of an exogenous nucleic acid comprising a nucleic acid sequence that controls or alters, e.g., increases, the expression of an endogenous nucleic acid sequence, e.g., endogenous gene. In such embodiments, the modified cell produces an endogenous polypeptide product that is naturally or endogenously expressed by the cell, but the modification increases the production of the product and/or the quality of the product as compared to an unmodified cell, e.g., as compared to endogenous production or quality of the polypeptide.

In another embodiment, the modification of the cell comprises the introduction of an exogenous nucleic acid encoding a recombinant polypeptide as described herein. In such embodiments, the modified cell produces a recombinant polypeptide product that can be naturally occurring or non-naturally occurring. In such embodiments, the modified cell produces a recombinant polypeptide product that can also be endogenously expressed by the cell or not. In embodiments where the recombinant polypeptide product is also endogenously expressed by the cell, the modification increases the production of the product and/or the quality of the product as compared to an unmodified cell, e.g., as compared to endogenous production or quality of the polypeptide.

As used herein, “recombinant polypeptide” or “recombinant protein” refers to a polypeptide that can be produced by a cell described herein. A recombinant polypeptide is one for which at least one nucleotide of the sequence encoding the polypeptide, or at least one nucleotide of a sequence which controls the expression of the polypeptide, was formed by genetic engineering (of the cell or of a precursor cell). E.g., at least one nucleotide was altered, e.g., it was introduced into the cell or it is the product of a genetically engineered rearrangement.

In an embodiment, the sequence of a recombinant polypeptide does not differ from a naturally occurring isoform of the polypeptide or protein. In an embodiment, the amino acid sequence of the recombinant polypeptide differs from the sequence of a naturally occurring isoform of the polypeptide or protein. In an embodiment, the recombinant polypeptide and the cell are from the same species. In an embodiment, the recombinant polypeptide is endogenous to the cell, in other words, the cell is from a first species and the recombinant polypeptide is native to that first species. In an embodiment, the amino acid sequence of the recombinant polypeptide is the same as or is substantially the same as, or differs by no more than 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% from, a polypeptide encoded by the endogenous genome of the cell. In an embodiment, the recombinant polypeptide and the cell are from different species, e.g., the recombinant polypeptide is a human polypeptide and the cell is a non-human, e.g., a rodent, e.g., a CHO, or an insect cell. In an embodiment, the recombinant polypeptide is exogenous to the cell, in other words, the cell is from a first species and the recombinant polypeptide is from a second species. In one embodiment, the polypeptide is a synthetic polypeptide. In one embodiment, the polypeptide is derived from a non-naturally occurring source. In an embodiment, the recombinant polypeptide is a human polypeptide or protein which does not differ in amino acid sequence from a naturally occurring isoform of the human polypeptide or protein. In an embodiment, the recombinant polypeptide differs from a naturally occurring isoform of the human polypeptide or protein at no more than 1, 2, 3, 4, 5, 10, 15 or 20 amino acid residues. In an embodiment, the recombinant polypeptide differs from a naturally occurring isoform of the human polypeptide by no more than 1, 2, 3, 4, 5, 6, 7, 8, 9, 10, or 15% of its amino acid residues.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, a “method of manufacturing” and a “method of production” are used interchangeably, and are a series of one or more operations and/or conditions that produces a sample comprising a product, e.g., a recombinant polypeptide or a therapeutic product.

As used herein, MS¹ means mass spectrometry.

As used herein, MS² means tandem mass spectrometry.

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

### Sample Preparation

Samples for use in the methods of the disclosure can be generated by many steps of methods of manufacturing and production of a product, e.g., a recombinant polypeptide. In some embodiments, a sample comprises one or more of culture supernatant, cell lysate, a product purification intermediate (e.g., a product partially purified from cellular proteins or other contaminants), a purified product, and a final formulated product (e.g., formulated for in vivo human use). The product comprised within a sample or generated by a method of manufacturing and production may be any product described herein, or known in the art.

### Chromatography

Methods of chromatography suitable for use in the methods described herein are known to one of skill in the art and include, e.g., affinity chromatography, gel filtration chromatography, ion exchange chromatography, reversed phase chromatography, hydrophobic interaction chromatography. In some embodiments, the chromatography method is HPLC reversed phase chromatography. Chromatography can include high performance liquid chromatography (HPLC), gas chromatography (GC), capillary electrophoresis, ion mobility. See also, e.g., Process Scale Purification of Antibodies, Uwe Gottschalk 2011 John Wiley & Sons ISBN: 1118210743; Antibodies Vol 1 Production and Purification, G. Subramanian 2013 Springer Science & Business Media; Basic Methods in Antibody Production and Characterization, Gary C. Howard 2000 CRC Press.

Additional exemplary chromatographic methods include, but are not limited to, Strong Anion Exchange chromatography (SAX), liquid chromatography (LC), high performance liquid chromatography (HPLC), ultra performance liquid chromatography (UPLC), thin layer chromatography (TLC), amide column chromatography, and combinations thereof.

In some embodiments, methods of the disclosure employ LC comprising one or more (e.g., one, two, or more) mobile phases and a stationary phase. In some embodiments, the LC comprises using one mobile phase. In some embodiments, the LC comprises using two mobile phases (e.g., a first mobile phase and a second mobile phase). In some embodiments, the mobile phase (e.g., a first and/or second mobile phase) comprises formic acid in water, e.g., about 0.01%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, or 1% formic acid in water. In some embodiments, the mobile phase (e.g., a first and/or second mobile phase) comprises formic acid in acetonitrile, e.g., about 0.01%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, or 1% formic acid in acetonitrile, e.g., 0.1% formic acid in acetonitrile. In some embodiments, “in acetonitrile” refers to a solution, e.g., mobile phase, wherein at least about 50, 55, 60, 65, 70, 75, 80, 85, 90, 95, or 100% of the solution, e.g., solvent, is acetonitrile, e.g., about 100% of the solvent is acetonitrile. In some embodiments, the stationary phase comprises a partially or fully fluorinated alkyl or aryl group, e.g., a fluorophenyl group, e.g., a pentafluorophenylpropyl group. In some embodiments, the stationary phase comprises a silica gel particle attached to a partially or fully fluorinated alkyl or aryl group, e.g., a fluorophenyl group, e.g., a pentafluorophenylpropyl group. In some embodiments, the stationary phase pore size is about 100, 110, 120, 130, 140, or 150 Å (e.g., 120 Å). In some embodiments, the LC comprises using a Discovery HS F5 stationary phase, e.g., a Discovery HS F5 column.

Without wishing to be bound by theory, it is thought that the partially or fully fluorinated alkyl or aryl group, e.g., a fluorophenyl group, e.g., pentafluorophenyl, coating of the column resin changes how tropolone is retained by the column. Whereas more traditional reverse phase columns were not sufficiently separating tropolone from interfering components, the partially or fully fluorinated alkyl or aryl group, e.g., a fluorophenyl group, e.g., pentafluorophenyl, resin coating is thought to retain hydrophobic groups more readily, and hydrophilic moieties elute more readily as a consequence.

### Mass Spectrometry

Mass spectrometry methods suitable for use in the methods described herein are known to one of skill in the art and include, e.g., electrospray ionization MS, matrix-assisted laser desportion/ionization MS (MALDI-MS), time of flight MS, fourier-transform ion cyclotron resonance MS, quadrupole time of flight MS, linear quadrupole, quadrupole ion trap MS, orbitrap, cylindrical ion trap, three dimensional ion trap, quadruple mass filter, tandem mass spectrometry, LC-MS, LC-MS/MS, Fourier transform mass spectrometry (FTMS), ion mobility separation with mass spectrometry (IMS-MS), electron transfer dissociation (ETD-MS), and combinations thereof. In some embodiments, the mass spectrometry is tandem mass spectrometry (MS²). See also, e.g., Protein Mass Spectrometry, Julian Whitelegge 2008, Elsevier; Protein Sequencing and Identification Using Tandem Mass Spectrometry, Michael Kinter 2005, John Wiley & Sons; Characterization of Protein Therapeutics using Mass Spectrometry, Guodong Chen 2014, Springer Science & Business Media.

In some embodiments, mass spectrometry suitable for use in the methods described herein comprises selected reaction monitoring (SRM), e.g., monitoring a selected precursor and product ion pair, e.g., transition. In some embodiments, mass spectrometry suitable for use in the methods described herein comprises multiple reaction monitoring (MRM), e.g., monitoring a plurality of product ions derived from one or more precursor ions, e.g., a plurality of transitions. In some embodiments, mass spectrometry suitable for use in the methods described herein comprises parallel reaction monitoring (PRM), e.g., monitoring a plurality of transitions in a single analysis step, e.g., using a high resolution mass spectrometer. In some embodiments, mass spectrometry suitable for use in the methods described herein comprises monitoring a transition recited in Table 1, e.g., under conditions recited in Table 1.

### Tropolone and Compounds Useful in Biomanufacturing

In some embodiments, a compound may be added to a cell culture medium to enhance cell growth. For example, the compound may be used to facilitate the uptake of metal ions in cultured cells. In some embodiments, compound added to a cell culture medium is a compound of Formula (I):

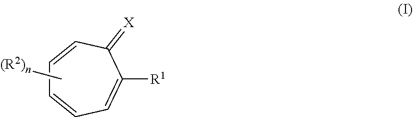

or a pharmaceutically acceptable salt, stereoisomer, racemate, or solvate thereof, wherein:

X is O or S;

R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;

each R² is independently C₁-C₆ alkyl, C₁-C₆ heteroalkyl, N(R^{4a})(R^{4b}), C(O)N(R^{4a})(R^{4b}), or N(R^{4a})C(O)R5; or

two R² are joined to form a heterocyclyl ring optionally substituted with one or more R⁶; or R¹ and R² are joined to form a heterocyclyl ring optionally substituted with one or more R⁶.

R³ is hydrogen, C₁-C₆ alkyl, or C₁-C₆ heteroalkyl;

R^{4a} and R^{4b} are independently hydrogen, C₁-C₆ alkyl, or C₁-C₆ heteroalkyl;

R⁵ is C₁-C₆ alkyl or C₁-C₆ heteroalkyl;

each R⁶ is independently C₁-C₆ alkyl, C₁-C₆ heteroalkyl, halo, oxo, or cyano; and

n is 0, 1, 2, 4, or 5.

In some embodiments, X is O. In some embodiments, R¹ is OR³ (e.g., OH). In some embodiments, n is 0. In some embodiments, the compound of Formula (I) is tropolone (i.e., 2-hydroxy-2,4,6-cycloheptatrien-1-one). In some embodiments, the compound of Formula (I) is

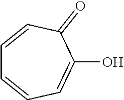

or a pharmaceutically acceptable salt thereof.

In some embodiments, X is O. In some embodiments, R¹ is OR³ (e.g., OH). In some embodiments, R² is OR³ or C(O)OR³ (e.g., OH or C(O)OH). In some embodiments, n is 3. In some embodiments, n is 3 and R² is OH, OH, and C(O)OH. In some embodiments, the compound of Formula (I) is puberulic acid (i.e., 4,5,6-trihydroxy-3-oxocyclohepta-1,4,6-triene-1-carboxylic acid). In some embodiments, the compound of Formula (I) is

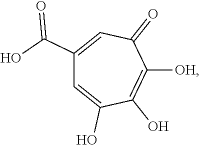 [SMILES: O=C(O)c1cc(O)c(O)c(O)c(=O)c1]

or a pharmaceutically acceptable salt thereof.

In some embodiments, X is O. In some embodiments, R¹ is hydrogen. In some embodiments, R² is OR³ or C(O)OR³ (e.g., OH or C(O)OH). In some embodiments, n is 3. In some embodiments, n is 3 and 2 R² are OH and 1 R² is C(O)OH. In some embodiments, the compound of Formula (I) is stipitatic acid (i.e., 5,6-dihydroxy-3-oxocyclohepta-1,4,6-triene-1-carboxylic acid). In some embodiments, the compound of Formula (I) is

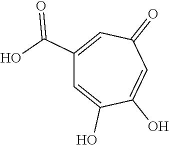 [SMILES: O=C(O)c1cc(O)c(O)cc(=O)c1]

or a pharmaceutically acceptable salt thereof.

In some embodiments, X is O. In some embodiments, R¹ is OR³ (e.g., OH). In some embodiments, R² is OR³, C(O)R⁵, or C(O)OR³ (e.g., OH or C(O)OH). In some embodiments, n is 3. In some embodiments, n is 3 and 1 R² is OH. In some embodiments, 2 R² are joined to form a heterocylyl ring (e.g., a 5-membered heterocylyl ring, e.g., maleic anhydride). In some embodiments, the compound of Formula (I) is stipitatonic acid (i.e., 4,7-dihydroxy-1H-cyclohepta[c]furan-1,3,6-trione). In some embodiments, the compound of Formula (I) is

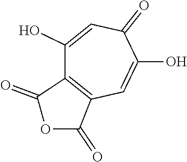 [SMILES: O=C1OC(=O)c2c(O)cc(=O)c(O)cc21]

or a pharmaceutically acceptable salt thereof.

In some embodiments, X is O. In some embodiments, R¹ is OR³ (e.g., OH). In some embodiments, R² is OR³, C(O)R⁵, or C(O)OR³ (e.g., OH or C(O)OH). In some embodiments, n is 3. In some embodiments, n is 4 and 2 R² are OH. In some embodiments, 2 R² are joined to form a heterocylyl ring (e.g., a 5-membered heterocylyl ring, e.g., succinic anhydride). In some embodiments, the compound of Formula (I) is puberulonic acid (i.e., 6,7,8-trihydroxy-1H-cyclohepta[c]furan-1,3,5-trione). In some embodiments, the compound of Formula (I) is

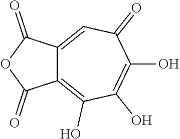 [SMILES: O=C1OC(=O)c2c1cc(=O)c(O)c(O)c2O]

or a pharmaceutically acceptable salt thereof.

In some embodiments, X is O. In some embodiments, R¹ is OR³ (e.g., OH). In some embodiments, R² is C₁-C₆ alkyl, C₁-C₆ heteroalkyl, or OR³ (e.g., OH). In some embodiments, n is 3. In some embodiments, n is 3 and 1 R² is OH. In some embodiments, 2 R² are joined to form a heterocylyl ring (e.g., a 6-membered heterocylyl ring, e.g., pyranyl ring) optionally substituted with one or more R⁶. In some embodiments, R⁶ is OR³ (e.g., OH) or C₁-C₆ alkyl (e.g., CH₃). In some embodiments, the compound of Formula (I) is sepedonin (i.e., 3,7,9-trihydroxy-3-methyl-3,4-dihydrocyclohepta[c]pyran-6(1H)-one). In some embodiments, the compound of Formula (I) is

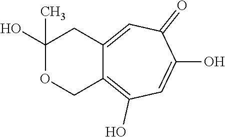 [SMILES: CC1(O)Cc2cc(=O)c(O)cc(O)c2CO1]

or a pharmaceutically acceptable salt thereof.

In some embodiments, the compound of Formula (I) is a compound disclosed in U.S. Pat. No. 3,135,768, which is incorporated herein by reference in its entirety.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972).

The term “alkyl,” as used herein, refers to a monovalent saturated, straight- or branched-chain hydrocarbon such as a straight or branched group of 1-12, 1-10, or 1-6 carbon atoms, referred to herein as C₁-C₁₂ alkyl, C₁-C₁₀ alkyl, and C₁-C₆ alkyl, respectively. Examples of alkyl groups include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

The term “heterocyclyl” refers to a monocyclic, or fused, spiro-fused, and/or bridged bicyclic and polycyclic ring system where at least one ring is saturated or partially unsaturated (but not aromatic) and comprises a heteroatom. A heterocyclyl can be attached to its pendant group at any heteroatom or carbon atom that results in a stable structure and any of the ring atoms can be optionally substituted. Representative heterocyclyls include ring systems in which (i) every ring is non-aromatic and at least one ring comprises a heteroatom, e.g., tetrahydrofuranyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl, 1,2,3,4-tetrahydroisoquinolinyl; and (iii) at least one ring is non-aromatic and comprises a heteroatom and at least one other ring is aromatic and comprises a heteroatom, e.g., 3,4-dihydro-1H-pyrano[4,3-c]pyridine, and 1,2,3,4-tetrahydro-2,6-naphthyridine

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N₊(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

### Production Parameters

The methods described herein can be used to analyze samples generated by methods of manufacturing and production, e.g., of recombinant polypeptides. The methods of manufacturing and production may be characterized by a variety of production parameters.

A production parameter as used herein is a parameter or element in a production process. Production parameters that can be selected include, e.g., the cell or cell line used to produce the glycoprotein preparation, the culture medium, culture process or bioreactor variables (e.g., batch, fed-batch, or perfusion), purification process and formulation of a glycoprotein preparation.

Primary production parameters include: 1) the types of host; 2) genetics of the host; 3) media type; 4) fermentation platform; 5) purification steps; and 6) formulation. Secondary production parameter, as used herein, is a production parameter that is adjustable or variable within each of the primary production parameters. Examples include: selection of host subclones based on desired glycan properties; regulation of host gene levels constitutive or inducible; introduction of novel genes or promoter elements; media additives (e.g. partial list on Table IV); physiochemical growth properties; growth vessel type (e.g. bioreactor type, T flask); cell density; cell cycle; enrichment of product with a desired glycan type (e.g. by lectin or antibody-mediated enrichment, ion-exchange chromatography, CE, or similar method); or similar secondary production parameters clear to someone skilled in the art.

Media

The methods of manufacturing and production described herein can include determining and/or selecting a media component and/or the concentration of a media component that has a positive correlation to a desired glycan property or properties. A media component can be added in or administered over the course of glycoprotein production or when there is a change media, depending on culture conditions. Media components include components added directly to culture as well as components that are a byproduct of cell culture.

Media components include, e.g., buffer, amino acid content, vitamin content, salt content, mineral content, serum content, carbon source content, lipid content, nucleic acid content, hormone content, trace element content, ammonia content, co-factor content, indicator content, small molecule content, hydrolysate content and enzyme modulator content.

Examples of various media components are provided below:

| | |
|---|---|
| amino acids | sugar precursors |
| Vitamins | Indicators |
| Carbon source (natural | Nucleosides or nucleotides |
| and unnatural) | |
| Salts | butyrate or organics |
| Sugars | DMSO |
| Sera | Animal derived products |
| Plant derived hydrolysates | Gene inducers |
| sodium pyruvate | Non natural sugars |
| Surfactants | Regulators of intracellular pH |
| Ammonia | Betaine or osmoprotectant |
| Lipids | Trace elements |
| Hormones or growth factors | minerals |
| Buffers | Non natural amino acids |
| Non natural amino acids | Non natural vitamins |

Exemplary buffers include Tris, Tricine, HEPES, MOPS, PIPES, TAPS, bicine, BES, TES, cacodylate, MES, acetate, MKP, ADA, ACES, glycinamide and acetamidoglycine. The media can be serum free or can include animal derived products such as, e.g., fetal bovine serum (FBS), fetal calf serum (FCS), horse serum (HS), human serum, animal derived serum substitutes (e.g., Ultroser G, SF and HY; non-fat dry milk; Bovine EX-CYTE), fetuin, bovine serum albumin (BSA), serum albumin, and transferrin. When serum free media is selected lipids such as, e.g., palmitic acid and/or steric acid, can be included.

Lipids components include oils, saturated fatty acids, unsaturated fatty acids, glycerides, steroids, phospholipids, sphingolipids and lipoproteins. Exemplary amino acid that can be included or eliminated from the media include alanine, arginine, asparagine, aspartic acid, cysteine, glutamic acid, glutamine, glycine, histidine, proline, isoleucine, leucine, lysine, methionine, phenylalanine, proline, serine, threonine, tryptophan, tyrosine and valine. Examples of vitamins that can be present in the media or eliminated from the media include vitamin A (retinoid), vitamin B1 (thiamine), vitamin B2 (riboflavin), vitamin B3 (niacin), vitamin B5 (pantothenic acid), vitamin B6 (pyroxidone), vitamin B7 (biotin), vitamin B9 (folic acid), vitamin. B12 (cyanocobalamin), vitamin C (ascorbic acid), vitamin D, vitamin E, and vitamin K.

Minerals that can be present in the media or eliminated from the media include bismuth, boron, calcium, chlorine, chromium, cobalt, copper, fluorine, iodine, iron, magnesium, manganese, molybdenum, nickel, phosphorus, potassium, rubidium, selenium, silicon, sodium, strontium, sulfur, tellurium, titanium, tungsten, vanadium, and zinc. Exemplary salts and minerals include CaCl2) (anhydrous), CuSO4 5H2O, Fe(NO3).9H2O, KCl, KNO3, KH2PO4, MgSO4 (anhydrous), NaCl, NaH2PO4H2O, NaHCO3, Na2SE3 (anhydrous), ZnSO4.7H2O; linoleic acid, lipoic acid, D-glucose, hypoxanthine 2Na, phenol red, putrescine 2HCl, sodium pyruvate, thymidine, pyruvic acid, sodium succinate, succinic acid, succinic acid.Na.hexahydrate, glutathione (reduced), para-aminobenzoic acid (PABA), methyl linoleate, bacto peptone G, adenosine, cytidine, guanosine, 2′-deoxyadenosine HCl, 2′-deoxycytidine HCl, 2′-deoxyguanosine and uridine. When the desired glycan characteristic is decreased fucosylation, the production parameters can include culturing a cell, e.g., CHO cell, e.g., dhfr deficient CHO cell, in the presence of manganese, e.g., manganese present at a concentration of about 0.1 μM to 50 μM. Decreased fucosylation can also be obtained, e.g., by culturing a cell (e.g., a CHO cell, e.g., a dhfr deficient CHO cell) at an osmolality of about 350 to 500 mOsm. Osmolality can be adjusted by adding salt to the media or having salt be produced as a byproduct as evaporation occurs during production.

Hormones include, for example, somatostatin, growth hormone-releasing factor (GRF), insulin, prolactin, human growth hormone (hGH), somatotropin, estradiol, and progesterone. Growth factors include, for example, bone morphogenic protein (BMP), epidermal growth factor (EGF), basic fibroblast growth factor (bFGF), nerve growth factor (NGF), bone derived growth factor (BDGF), transforming growth factor-beta1) (TGF-beta1), [Growth factors from U.S. Pat. No. 6,838,284 B2], hemin and NAD. Examples of surfactants that can be present or eliminated from the media include Tween-80 and pluronic F-68. Small molecules can include, e.g., butyrate, ammonia, non natural sugars, non natural amino acids, chloroquine, and betaine.

Physiochemical Parameters

Production parameters can also include physiochemical parameters. Such conditions can include temperature, pH, osmolality, shear force or agitation rate, oxidation, spurge rate, growth vessel, tangential flow, DO, CO₂, nitrogen, fed batch, redox, cell density and feed strategy. Examples of physiochemical parameters that can be selected include, e.g., pH, osmolality, shear force or agitation rate, oxidation, spurge rate, growth vessel, tangential flow, batch dissolved O2, CO₂, nitrogen, fed batch, redox, cell density, perfusion culture, feed strategy, temperature and time of culture.

Additional production parameters are known to one of skill in the art, see e.g., Antibody Expression and Production (2011) Ed. Mohamed Al-Rubeai; Springer Publishing.

Products and Nucleic Acids Encoding them

Provided herein are methods of analyzing samples, e.g., samples produced by methods of manufacturing and production, e.g., of recombinant polypeptides. The methods of manufacturing and production may comprise identifying, selecting, or making a cell or cell line capable of producing a product, e.g., cells and products as recited herein. The products encompassed by the present disclosure include, but are not limited to, molecules, nucleic acids, polypeptides (e.g., recombinant polypeptides, e.g., antibodies, bispecific antibodies, multispecific antibodies), or hybrids thereof, that can be produced by, e.g., expressed in, a cell. In some embodiments, the cells are engineered or modified to produce the product. Such modifications include the introducing molecules that control or result in production of the product. For example, a cell is modified by introducing an exogenous nucleic acid that encodes a polypeptide, e.g., a recombinant polypeptide, and the cell is cultured under conditions suitable for production, e.g., expression and secretion, of the polypeptide, e.g., recombinant polypeptide.

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

In embodiments, the polypeptide is, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide, calcitonin, etanercept, hemoglobin glutamer 250 (bovine), drotrecogin alpha, collagenase, carperitide, recombinant human epidermal growth factor, DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (Hydron), deslorelin, histrelin, nafarelin, leuprolide (ATRIGEL), leuprolide (DUROS), goserelin, Eutropin, somatropin, mecasermin, enlfavirtide, Org-33408, insulin glargine, insulin glulisine, insulin (inhaled), insulin lispro, insulin deternir, insulin (RapidMist), mecasermin rinfabate, anakinra, celmoleukin, 99 mTc-apcitide, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex’ recombinant human luteinizing hormone, dornase alpha, trafermin, ziconotide, taltirelin, diboterminalfa, atosiban, becaplermin, eptifibatide, Zemaira, CTC-111, Shanvac-B, octreotide, lanreotide, ancestirn, agalsidase beta, agalsidase alpha, laronidase, prezatide copper acetate, rasburicase, ranibizumab, Actimmune, PEG-Intron, Tricomin, recombinant human parathyroid hormone (PTH) 1-84, epoetin delta, transgenic antithrombin III, Granditropin, Vitrase, recombinant insulin, interferon-alpha, GEM-21S, vapreotide, idursulfase, omnapatrilat, recombinant serum albumin, certolizumab pegol, glucarpidase, human recombinant C1 esterase inhibitor, lanoteplase, recombinant human growth hormone, enfuvirtide, VGV-1, interferon (alpha), lucinactant, aviptadil, icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide, tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone, recombinant G-CSF, insulin, insulin (Technosphere), insulin (AERx), RGN-303, DiaPep277, interferon beta, interferon alpha-n3, belatacept, transdermal insulin patches, AMG-531, MBP-8298, Xerecept, opebacan, AIDSVAX, GV-1001, LymphoScan, ranpirnase, Lipoxysan, lusupultide, MP52, sipuleucel-T, CTP-37, Insegia, vitespen, human thrombin, thrombin, TransMID, alfimeprase, Puricase, terlipressin, EUR-1008M, recombinant FGF-I, BDM-E, rotigaptide, ETC-216, P-113, MBI-594AN, duramycin, SCV-07, OPI-45, Endostatin, Angiostatin, ABT-510, Bowman Birk Inhibitor, XMP-629, 99 mTc-Hynic-Annexin V, kahalalide F, CTCE-9908, teverelix, ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, SB-249553, Oncovax-CL, OncoVax-P, BLP-25, CerVax-16, MART-1, gp100, tyrosinase, nemifitide, rAAT, CGRP, pegsunercept, thymosinbeta4, plitidepsin, GTP-200, ramoplanin, GRASPA, OBI-1, AC-100, salmon calcitonin (eligen), examorelin, capromorelin, Cardeva, velafermin, 131I-TM-601, KK-220, T-10, ularitide, depelestat, hematide, Chrysalin, rNAPc2, recombinant Factor V111 (PEGylated liposomal), bFGF, PEGylated recombinant staphylokinase variant, V-10153, SonoLysis Prolyse, NeuroVax, CZEN-002, rGLP-1, BIM-51077, LY-548806, exenatide (controlled release, Medisorb), AVE-0010, GA-GCB, avorelin, ACM-9604, linaclotid eacetate, CETi-1, Hemospan, VAL, fast-acting insulin (injectable, Viadel), insulin (eligen), recombinant methionyl human leptin, pitrakinra, Multikine, RG-1068, MM-093, NBI-6024, AT-001, PI-0824, Org-39141, Cpn10, talactoferrin, rEV-131, rEV-131, recombinant human insulin, RPI-78M, oprelvekin, CYT-99007 CTLA4-Ig, DTY-001, valategrast, interferon alpha-n3, IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, ALTU-135, recombinant neuraminidase, Vacc-5q, Vacc-4x, Tat Toxoid, YSPSL, CHS-13340, PTH(1-34) (Novasome), Ostabolin-C, PTH analog, MBRI-93.02, MTB72F, MVA-Ag85A, FARA04, BA-210, recombinant plague FIV, AG-702, OxSODrol, rBetV1, Der-p1/Der-p2/Der-p7, PR1 peptide antigen, mutant ras vaccine, HPV-16 E7 lipopeptide vaccine, labyrinthin, WT1-peptide, IDD-5, CDX-110, Pentrys, Norelin, CytoFab, P-9808, VT-111, icrocaptide, telbermin, rupintrivir, reticulose, rGRF, HA, alpha-galactosidase A, ACE-011, ALTU-140, CGX-1160, angiotensin, D-4F, ETC-642, APP-018, rhMBL, SCV-07, DRF-7295, ABT-828, ErbB2-specific immunotoxin, DT3SSIL-3, TST-10088, PRO-1762, Combotox, cholecystokinin-B/gastrin-receptor binding peptides, 111In-hEGF, AE-37, trasnizumab-DM1, Antagonist G, IL-12, PM-02734, IMP-321, rhIGF-BP3, BLX-883, CUV-1647, L-19 based ra, Re-188-P-2045, AMG-386, DC/1540/KLH, VX-001, AVE-9633, AC-9301, NY-ESO-1 (peptides), NA17.A2 peptides, CBP-501, recombinant human lactoferrin, FX-06, AP-214, WAP-8294A, ACP-HIP, SUN-11031, peptide YY [3-36], FGLL, atacicept, BR3-Fc, BN-003, BA-058, human parathyroid hormone 1-34, F-18-CCR1, AT-1100, JPD-003, PTH(7-34) (Novasome), duramycin, CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix, EP-51216, hGH, OGP-I, sifuvirtide, TV4710, ALG-889, Org-41259, rhCC10, F-991, thymopentin, r(m)CRP, hepatoselective insulin, subalin, L19-IL-2 fusion protein, elafin, NMK-150, ALTU-139, EN-122004, rhTPO, thrombopoietin receptor agonist, AL-108, AL-208, nerve growth factor antagonists, SLV-317, CGX-1007, INNO-105, teriparatide (eligen), GEM-OS1, AC-162352, PRX-302, LFn-p24 fusion, EP-1043, gpE1, gpE2, MF-59, hPTH(1-34), 768974, SYN-101, PGN-0052, aviscumnine, BIM-23190, multi-epitope tyrosinase peptide, enkastim, APC-8024, GI-5005, ACC-001, TTS-CD3, vascular-targeted TNF, desmopressin, onercept, and TP-9201.

In some embodiments, the polypeptide is adalimumab (HUMIRA), infliximab (REMICADE™), rituximab (RITUXAN™/MAB THERA™) etanercept (ENBREL™) bevacizumab (AVASTIN™), trastuzumab (HERCEPTIN™), pegrilgrastim (NEULASTA™), or any other suitable polypeptide including biosimilars and biobetters.

Other suitable polypeptides are those listed below and in Table 1 of US2016/0097074:

TABLE 1

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human leukocyte derived | Alferon N ® |
| human antihemophilic factor | Alphanate ® |
| virus-filtered human coagulation factor IX | AlohaNine ® SD |
| Alefacept; recombinant, dimeric | Amevive ® |
| fusion protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| interferon beta-lb | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab: technetium-99 labeled | CEA-Scan ® |
| alglucerase; modified form of | Ceredase ® |
| beta-glucocerebrosidase | |
| imiglucerase; recombinant form of | Cerezyme ® |
| beta-glucocerebrosidase | |
| crotalidae polyvalent immune Fab, ovine | CroFab ™ |
| digoxin Immune fab [ovine] | DigiFab ™ |
| Rasburicase | Elitek ® |
| Etanercept | ENBREL ® |
| epoletin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von | Humate-P ® |
| Willebrand factor complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony- | Leukine ®/Leukine ® Liquid |
| stimulating factor | |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesemab | NeutroSpec ™ (formerly LeuTech ®) |
| somatropin [rDNA] | Norditropin ®/Norditropin Nordiflex ® |
| Mitoxantrone | Novantrone ® |
| insulin:, zinc suspension; | Novolin L ® |
| insulin; isophane suspension | Novolin N ® |
| insulin. regular; | Novolin R ® |
| Insulin | Novolin ® |
| coagulation factor VIIa | NovoSeven ® |
| Somatropin | Nutropin ® |
| immunoglobulin intravenous | Octagame ® |
| PEG-L-asparaginase | Oncaspar ® |
| abatacept, fuIly human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| abatacept, fully human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); | Plenaxis ™ |
| gonadotropin-releasing hormone antagonist | |
| eboietin alpha | Procrit ® |
| Aldesleukin | Proleukin IL-2 ® |
| Somatrem | Protropin ® |
| dornase alfa | Pulmozyme ® |
| Efalizumab; selective, | RAPTIVA ™ |
| reversible T-cell blocker | |
| combination of ribavirin | Rebetron ™ |
| and alpha interferon | |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Ecuilzumab | SOLIRIS (R) |
| Pegvisomant | SOMAVERT ® |
| Pailvisumab; recombinantly | Synagis ™ |
| produced, humanized mAb | |
| thyrotropin alfa | Thyrogen ® |
| Tenectepiase | TNkase ™ |
| Natalizumab | TYSABRI ® |
| human immune globulin intravenous | Venoglobulin-S ® |
| 5% and 10% solutions | |
| interferon alfa-n1, lymphoblastoid | Wellferon ® |
| drotrecogin alfa | Xigris ™ |
| Omalizumab; recombinant DNA- | Xolair ® |
| derived humanized monoclonal | |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In embodiments, the polypeptide is a hormone, blood clotting/coagulation factor, cytokine/growth factor, antibody molecule, fusion protein, protein vaccine, or peptide as shown in Table 2.

TABLE 2
Exemplary Products

| Therapeutic | | |
|---|---|---|
| Product type | Product | Trade Name |
| Hormone | Erythropoietin, Epoein-a | Epogen, Procrit |
| | Darbepoetin-α | Aranesp |
| | Growth hormone (GH), | Genotropin, Humatrope, |
| | somatotropin | Norditropin, |
| | Human follicle-stimulating | NovIVitropin, |
| | hormone (FSH) | Nutropin, Omnitrope, |
| | Human chorionic | Protropin, Siazen, |
| | gonadotropin | Serostim, Valtropin |
| | Lutropin-α | Gonal-F, Follistim |
| | Glucagon | Ovidrel |
| | Growth hormone releasing | Luveris |
| | hormone (GHRH) | GlcaGen |
| | Secretin | Geref |
| | Thyroid stimulating | ChiRhoStim (human peptide), |
| | hormone (TSH), thyrotropin | SecreFlo (porcine peptide) |
| | | Thyrogen |
| Blood | Factor VIIa | NovoSeven |
| Clotting/ | Factor VIII | Bioclate, Helixate, Kogenate, |
| Coagulation | Factor IX | Recombinate, ReFacto |
| Factors | Antithrombin III (AT-III) | Benefix |
| | Protein C concentrate | Thrombate III |
| | | Ceprotin |
| Cytokine/ | Type I alpha-interferon | Infergen |
| Growth | Interferon-αn3 (IFNαn3) | Alferon N |
| factor | Interferon-β1a (rIFN-β) | Avonex, Rebif |
| | Interferon-β1b (rIFN-β) | Betaseron |
| | Interferon-γ1b (IFN γ) | Actimmune |
| | Aldesleukin (interleukin | Proleukin |
| | 2 (IL2), epidermal | Kepivance |
| | theymocyte activating | Regranex |
| | factor; ETAF | Anril, Kineret |
| | Palifermin (keratinocyte | |
| | growth factor; KGF) | |
| | Becaplemin (platelet- | |
| | derived growth factor; | |
| | PDGF) | |
| | Anakinra (recombinant | |
| | IL1 antagonist) | |
| Antibody | Bevacizumab | Avastin |
| molecules | (VEGFA mAb) | Erbitux |
| | Cetuximab (EGFR mAb) | Vectibix |
| | Panitumumab (EGFR mAb) | Campath |
| | Alemtuzumab (CD52 mAb) | Rituxan |
| | Rituximab (CD20 | Herceptin |
| | chimeric Ab) | Orencia |
| | Trastuzumab (HER2/ | Humira |
| | NeumAb) | Enbrel |
| | Abatacept (CTLA | Remicade |
| | Ab/Fc fusion) | Amevive |
| | Adalimumab (TNFα mAb) | Raptiva |
| | Etanercept (TNF | Tysabri |
| | receptor/Fc fusion) | Soliris |
| | Infliximab (TNFα | Orthoclone, OKT3 |
| | chimeric mAb) | |
| | Alefacept (CD2 | |
| | fusion protein) | |
| | Efalizumab (CD1la mAb) | |
| | Natalizumab (integrin α4 | |
| | subunit mAb) | |
| | Eculizumab (C5mAb) | |
| | Muromonab-CD3 | |
| Other: | Insulin | Humulin, Novolin |
| Fusion | Hepatitis B surface | Engerix, Recombivax HB |
| proteins/ | antigen (HBsAg) | Gardasil |
| Protein | HPV vaccine | LYMErix |
| vaccines/ | OspA | Rhophylac |
| Peptides | Anti-Rhesus (Rh) | Fuzeon |
| | immunoglobulin G | QMONOS |
| | Enfuvirtide | |
| | Spider silk, e.g., fibrion | |

In embodiments, the protein is a multispecific protein, e.g., a bispecific antibody as shown in Table 3.

TABLE 3
Bispecific Formats

| Name | | | | | |
|---|---|---|---|---|---|
| (other names, | | | Proposed | | Diseases (or |
| sponsoring | BsAb | | mechanisms of | Development | healthy |
| organizations) | format | Targets | action | stages | volunteers) |
| Catumaxomab | BsIgG: | CD3, | Retargeting of T | Approved in | Malignant ascites |
| (Removab ®, | Triomab | EpCAM | cells to tumor, Fc | EU | in EpCAM |
| Fresenius Biotech, | | | mediated effector | | positive tumors |
| Trion Pharma, | | | functions | | |
| Neopharm) | | | | | |
| Ertumaxomab | BsIgG: | CD3, HER2 | Retargeting of T | Phase I/II | Advanced solid |
| (Neovii Biotech, | Triomab | | cells to tumor | | tumors |
| Fresenius Biotech) | | | | | |
| Blinatumomab | BiTE | CD3, CD19 | Retargeting of T | Approved in | Precursor B-cell |
| (Blincyto ®, AMG | | | cells to tumor | USA | ALL |
| 103, MT 103, | | | | Phase II | ALL |
| MEDI 538, | | | | and III | DLBCL |
| Amgen) | | | | Phase II | NHL |
| | | | | Phase I | |
| REGN1979 | BsAb | CD3, CD20 | | | |
| (Regeneron) | | | | | |
| Solitomab (AMG | BiTE | CD3, | Retargeting of T | Phase I | Solid tumors |
| 110, MT110, | | EpCAM | cells to tumor | | |
| Amgen) | | | | | |
| MEDI 565 (AMG | BiTE | CD3, CEA | Retargeting of T | Phase I | Gastrointestinal |
| 211, MedImmune, | | | cells to tumor | | adenocancinoma |
| Amgen) | | | | | |
| RO6958688 | BsAb | CD3, CEA | | | |
| (Roche) | | | | | |
| BAY2010112 | BiTE | CD3, PSMA | Retargeting of T | Phase I | Prostate cancer |
| (AMG 212, Bayer; | | | cells to tumor | | |
| Amgen) | | | | | |
| MGD006 | DART | CD3, CD123 | Retargeting of T | Phase I | AML |
| (Macrogenics) | | | cells to tumor | | |
| MGD007 | DART | CD3, gpA33 | Retargeting of T | Phase I | Colorectal |
| (Macrogenics) | | | cells to tumor | | cancer |
| MGD011 | DART | CD19, CD3 | | | |
| (Macrogenics) | | | | | |
| SCORPION | BsAb | CD3, CD19 | Retargeting of T | | |
| (Emergent | | | cells to tumor | | |
| Biosolutions, | | | | | |
| Trubion) | | | | | |
| AFM11 (Affimed | TandAb | CD3, CD19 | Retargeting of T | Phase I | NHL and ALL |
| Therapeutics) | | | cells to tumor | | |
| AFM12 (Affimed | TandAb | CD19, CD16 | Retargeting of NK | | |
| Therapeutics) | | | cells to tumor | | |
| | | | cells | | |
| AFM13 (Affimed | TandAb | CD30, | Retargeting of NK | Phase II | Hodgkin's |
| Therapeutics) | | CD16A | cells to tumor | | Lymphoma |
| | | | cells | | |
| GD2 (Barbara Ann | T cells | CD3, GD2 | Retargeting of T | Phase I/II | Neuroblastoma |
| Karmanos Cancer | preloaded | | cells to tumor | | and |
| Institute) | with BsAb | | | | osteosarcoma |
| pGD2 (Barbara | T cells | CD3, Her2 | Retargeting of T | Phase II | Metastatic |
| Ann Karmanos | preloaded | | cells to tumor | | breast |
| Cancer Institute) | with BsAb | | | | cancer |
| EGFRBi-armed | T cells | CD3, EGFR | Autologous | Phase I | Lung and other |
| autologous | preloaded | | activated T cells | | solid tumors |
| activated T cells | with BsAb | | to EGFR-positive | | |
| (Roger Williams | | | tumor | | |
| Medical Center) | | | | | |
| Anti-EGFR-armed | T cells | CD3, EGFR | Autologous | Phase I | Colon and |
| activated T-cells | preloaded | | activated T cells | | pancreatic |
| (Barbara Ann | with BsAb | | to EGFR-positive | | cancers |
| Karmanos Cancer | | | tumor | | |
| Institute) | | | | | |
| rM28 (University | Tandem | CD28, | Retargeting of T | Phase II | Metastatic |
| Hospital Tübingen) | scFv | MAPG | cells to tumor | | melanoma |
| IMCgp100 | ImmTAC | CD3, peptide | Retargeting of T | Phase I/II | Metastatic |
| (Immunocore) | | MHC | cells to tumor | | melanoma |
| DT2219ARL | 2 scFv | CD19, CD22 | Targeting of | Phase I | B cell leukemia |
| (NCI, University of | linked to | | protein toxin to | | or lymphoma |
| Minnesota) | diphtheria | | tumor | | |
| | toxin | | | | |
| XmAb5871 | BsAb | CD19, | | | |
| (Xencor) | | CD32b | | | |
| NI-1701 | BsAb | CD47, CD19 | | | |
| (NovImmune) | | | | | |
| MM-111 | BsAb | ErbB2, | | | |
| (Merrimack) | | ErbB3 | | | |
| MM-141 | BsAb | IGF-1R, | | | |
| (Merrimack) | | ErbB3 | | | |
| NA (Merus) | BsAb | HER2, | | | |
| | | HER3 | | | |
| NA (Merus) | BsAb | CD3, | | | |
| | | CLEC12A | | | |
| NA (Merus) | BsAb | EGFR, | | | |
| | | HER3 | | | |
| NA (Merus) | BsAb | PD1, | | | |
| | | undisclosed | | | |
| NA (Merus) | BsAb | CD3, | | | |
| | | undisclosed | | | |
| Duligotuzumab | DAF | EGFR, | Blockade of 2 | Phase I and II | Head and neck |
| (MEHD7945A, | | HER3 | receptors, ADCC | Phase II | cancer |
| Genentech, Roche) | | | | | Colorectal cancer |
| LY3164530 (Eli | Not | EGFR, MET | Blockade of 2 | Phase I | Advanced or |
| Lily) | disclosed | | receptors | | metastatic cancer |
| MM-111 | HSA body | HER2, | Blockade of 2 | Phase II | Gastric and |
| (Merrimack | | HER3 | receptors | Phase I | esophageal |
| Pharmaceuticals) | | | | | cancers |
| | | | | | Breast cancer |
| MM-141, | IgG-scFv | IGF-1R, | Blockade of 2 | Phase I | Advanced solid |
| (Merrimack | | HER3 | receptors | | tumors |
| Pharmaceuticals) | | | | | |
| RG7221 | CrossMab | Ang2, | Blockade of 2 | Phase I | Solid tumors |
| (RO5520985, | | VEGFA | proangiogenics | | |
| Roche) | | | | | |
| RG7716 (Roche) | CrossMab | Ang2, | Blockade of 2 | Phase I | Wet AMD |
| | | VEGFA | proangiogemcs | | |
| OMP-305B83 | BsAb | DLL4/VEGF | | | |
| (OncoMed) | | | | | |
| TF2 | Dock and | CEA, HSG | Pretargeting | Phase II | Colorectal, |
| (Immunomedics) | lock | | tumor for PET or | | breast and lung |
| | | | radioimaging | | cancers |
| ABT-981 | DVD-Ig | IL-1α, IL-1β | Blockade of 2 | Phase II | Osteoarthritis |
| (AbbVie) | | | proinflammatory | | |
| | | | cytokines | | |
| ABT-122 | DVD-Ig | TNF, IL-17A | Blockade of 2 | Phase II | Rheumatoid |
| (AbbVie) | | | proinflammatory | | arthritis |
| | | | cytokines | | |
| COVA322 | IgG- | TNF, IL17A | Blockade of 2 | Phase I/II | Plaque psoriasis |
| | fynomer | | proinflammatory | | |
| | | | cytokines | | |
| SAR156597 | Tetravalent | IL-13, IL-4 | Blockade of 2 | Phase I | Idiopathic |
| (Sanofi) | bispecific | | proinflammatory | | pulmonary |
| | tandem IgG | | cytokines | | fibrosis |
| GSK2434735 | Dual- | IL-13, IL-4 | Blockade of 2 | Phase I | (Healthy |
| (GSK) | targeting | | proinflammatory | | volunteers) |
| | domain | | cytokines | | |
| Ozoralizumab | Nanobody | TNF, HSA | Blockade of | Phase II | Rheumatoid |
| (ATN103, | | | proinflammatory | | arthritis |
| Ablynx) | | | cytokine, binds to | | |
| | | | HSA to increase | | |
| | | | half-life | | |
| ALX-0761 | Nanobody | IL-17A/F, | Blockade of 2 | Phase I | (Healthy |
| (Merck | | HSA | proinflammatory | | volunteers) |
| Serono, | | | cytokines, binds | | |
| Ablynx) | | | to HSA to | | |
| | | | increase half-life | | |
| ALX-0061 | Nanobody | IL-6R, HSA | Blockade of | Phase I/II | Rheumatoid |
| (AbbVie, | | | proinflammatory | | arthritis |
| Ablynx; | | | cytokine, binds to | | |
| | | | HSA to increase | | |
| | | | half-life | | |
| ALX-0141 | Nanobody | RANKL, | Blockade of bone | Phase I | Postmenopausal |
| (Ablynx, | | HSA | resorption, binds | | bone loss |
| Eddingpharm) | | | to HSA to | | |
| | | | increase half-life | | |
| RG6013/ACE910 | ART-Ig | Factor IXa, | Plasma | Phase II | Hemophilia |
| (Chugai, Roche) | | factor X | coagulation | | |

TABLE 4

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human | Alferon N ® |
| leukocyte derived | |
| human antihemophilic factor | Alphanate ® |
| virus-filtered human coagulation | AlphaNine ® SD |
| factor IX | |
| Alefacept; recombinant, dimeric | Amevive ® |
| fusion protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form | Cerezyme ® |
| of beta-glucocerebrosidase | |
| crotalidae polyvalent immune | CroFab ™ |
| Fab, ovine | |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von | Humate-P ® |
| Willebrand factor complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony- | Leukine ®/Leukine ® |
| stimulating factor | Liquid |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly |
| | LeuTech ®) |
| somatropin [rDNA] | Norditropin ®/Norditropin |
| | Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human | Orencia ™ |
| soluable fusion protein | |
| muromomab-CD3 | Orthoclone OKT3 ® |
| high-molecular weight hyaluronan | Orthovisc ® |
| human chorionic gonadotropin | Ovidrel ® |
| live attenuated Bacillus | Pacis ® |
| Calmette-Guerin | |
| peginterferon alfa-2a | Pegasys ® |
| pegylated version of | PEG-Intron ™ |
| interferon alfa-2b | |
| Abarelix (injectable suspension); | Plenaxis ™ |
| gonadotropin-releasing hormone | |
| Antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, | RAPTIVA ™ |
| reversible T-cell blocker | |
| combination of ribavirin | Rebetron ™ |
| and alpha interferon | |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly | Synagis ™ |
| produced, humanized mAb | |
| thyrotropin alfa | Thyrogen ® |
| Tenecteplase | TNKase ™ |
| Natalizumab | TYSABRI ® |
| human immune globulin | Venoglobulin-S ® |
| intravenous 5% and 10% solutions | |
| interferon alfa-n1, lymphoblastoid | Wellferon ® |
| drotrecogin alfa | Xigris ™ |
| Omalizumab; recombinant DNA- | Xolair ® |
| derived humanized monoclonal | |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In some embodiments, the polypeptide is an antigen expressed by a cancer cell. In some embodiments the recombinant or therapeutic polypeptide is a tumor-associated antigen or a tumor-specific antigen. In some embodiments, the recombinant or therapeutic polypeptide is selected from HER2, CD20, 9-O-acetyl-GD3, PhCG, A33 antigen, CA19-9 marker, CA-125 marker, calreticulin, carboanhydrase IX (MN/CA IX), CCR5, CCR8, CD19, CD22, CD25, CD27, CD30, CD33, CD38, CD44v6, CD63, CD70, CC123, CD138, carcinoma embryonic antigen (CEA; CD66e), desmoglein 4, E-cadherin neoepitope, endosialin, ephrin A2 (EphA2), epidermal growth factor receptor (EGFR), epithelial cell adhesion molecule (EpCAM), ErbB2, fetal acetylcholine receptor, fibroblast activation antigen (FAP), fucosyl GM1, GD2, GD3, GM2, ganglioside GD3, Globo H, glycoprotein 100, HER2/neu, HER3, HER4, insulin-like growth factor receptor 1, Lewis-Y, LG, Ly-6, melanoma-specific chondroitin-sulfate proteoglycan (MCSCP), mesothelin, MUCl, MUC2, MUC3, MUC4,

MUC5_{Ac}, MUC5_{B}, MUC7, MUC16, Mullerian inhibitory substance (MIS) receptor type II, plasma cell antigen, poly SA, PSCA, PSMA, sonic hedgehog (SHH), SAS, STEAP, sTn antigen, TNF-alpha precursor, and combinations thereof.

In some embodiments, the polypeptide is an activating receptor and is selected from 2B4 (CD244), α₄β₁ integrin, β₂ integrins, CD2, CD16, CD27, CD38, CD96, CD100, CD160, CD137, CEACAMl (CD66), CRTAM, CSl (CD319), DNAM-1 (CD226), GITR (TNFRSF18), activating forms of KIR, NKG2C, NKG2D, NKG2E, one or more natural cytotoxicity receptors, NTB-A, PEN-5, and combinations thereof, optionally wherein the β₂ integrins comprise CD11a-CD 18, CD11 b-CD 18, or CD11c-CD 18, optionally wherein the activating forms of KIR comprise KlR2DSl, KIR2DS4, or KIR-S, and optionally wherein the natural cytotoxicity receptors comprise NKp30, NKp44, NKp46, or NKp80.

In some embodiments, the polypeptide is an inhibitory receptor and is selected from KIR, ILT2/LIR-l/CD85j, inhibitory forms of KIR, KLRG1, LAIR-1, NKG2A, NKR-PA, Siglec-3, Siglec-7, Siglec-9, and combinations thereof, optionally wherein the inhibitory forms of KIR comprise KIR2DL1, KIR2DL2, KIR2DL3, KIR3DL1, KIR3DL2, or KIR-L.

In some embodiments, the polypeptide is an activating receptor and is selected from CD3, CD2 (LFA2, OX34), CD5, CD27 (TNFRSF7), CD28, CD30 (TNFRSF8), CD40L, CD84 (SLAMF5), CD137 (4-1BB), CD226, CD229 (Ly9, SLAMF3), CD244 (2B4, SLAMF4), CD319 (CRACC, BLAME), CD352 (Lyl08, NTBA, SLAMF6), CRTAM (CD355), DR3 (TNFRSF25), GITR (CD357), HVEM (CD270), ICOS, LIGHT, LTβR (TNFRSF3), OX40 (CD134), NKG2D, SLAM (CD150, SLAMF1), TCRα, TCRβ, TCRδγ, TIM1 (HAVCR, KIM1), and combinations thereof.

In some embodiments, the polypeptide is an inhibitory receptor and is selected from PD-1 (CD279), 2B4 (CD244, SLAMF4), B71 (CD80), B7Hl (CD274, PD-L1), BTLA (CD272), CD160 (BY55, NK28), CD352 (Ly108, NTBA, SLAMF6), CD358 (DR6), CTLA-4 (CD152), LAG3, LAIR1, PD-1H (VISTA), TIGIT (VSIG9, VSTM3), TIM2 (TIMD2), TIM3 (HAVCR2, KIM3), and combinations thereof.

Other exemplary proteins include, but are not limited to any protein described in Tables 1-10 of Leader et al., “Protein therapeutics: a summary and pharmacological classification”, Nature Reviews Drug Discovery, 2008, 7:21-39 (incorporated herein by reference); or any conjugate, variant, analog, or functional fragment of the recombinant polypeptides described herein.

Other recombinant protein products include non-antibody scaffolds or alternative protein scaffolds, such as, but not limited to: DARPins, affibodies and adnectins. Such non-antibody scaffolds or alternative protein scaffolds can be engineered to recognize or bind to one or two, or more, e.g., 1, 2, 3, 4, or 5 or more, different targets or antigens.

Also provided herein are nucleic acids, e.g., exogenous nucleic acids that encode the products, e.g., polypeptides, e.g., recombinant polypeptides described herein. The nucleic acid sequences coding for the desired recombinant polypeptides can be obtained using recombinant methods known in the art, such as, for example by screening libraries from cells expressing the desired nucleic acid sequence, e.g., gene, by deriving the nucleic acid sequence from a vector known to include the same, or by isolating directly from cells and tissues containing the same, using standard techniques. Alternatively, the nucleic acid encoding the recombinant polypeptide can be produced synthetically, rather than cloned. Recombinant DNA techniques and technology are highly advanced and well established in the art. Accordingly, the ordinarily skilled artisan having the knowledge of the amino acid sequence of a recombinant polypeptide described herein can readily envision or generate the nucleic acid sequence that would encode the recombinant polypeptide.

In some embodiments, the exogenous nucleic acid controls the expression of a product that is endogenously expressed by the host cell. In such embodiments, the exogenous nucleic acid comprises one or more nucleic acid sequences that increase the expression of the endogenous product (also referred to herein as “endogenous product transactivation sequence”). For example, the nucleic acid sequence that increases the expression of an endogenous product comprises a constitutively active promoter or a promoter that is stronger, e.g., increases transcription at the desired site, e.g., increases expression of the desired endogenous gene product. After introduction of the exogenous nucleic acid comprising the endogenous product transactivation sequence, said exogenous nucleic acid is integrated into the chromosomal genome of the cell, e.g., at a preselected location proximal to the genomic sequence encoding the endogenous product, such that the endogenous product transactivation sequence increases the transactivation or expression of the desired endogenous product. Other methods for modifying a cell, e.g., introducing an exogenous nucleic acid, for increasing expression of an endogenous product is described, e.g., in U.S. Pat. No. 5,272,071; hereby incorporated by reference in its entirety.

The expression of a product described herein is typically achieved by operably linking a nucleic acid encoding the recombinant polypeptide or portions thereof to a promoter, and incorporating the construct into an expression vector. The vectors can be suitable for replication and integration eukaryotes or prokaryotes. Typical cloning vectors contain other regulatory elements, such as transcription and translation terminators, initiation sequences, and promoters useful for regulation of the expression of the desired nucleic acid sequence.

The nucleic acid sequences described herein encoding a product, e.g., a recombinant polypeptide, or comprising a nucleic acid sequence that can control the expression of an endogenous product, can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In embodiments, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193). Vectors derived from viruses are suitable tools to achieve long-term gene transfer since they allow long-term, stable integration of a transgene and its propagation in daughter cells.

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection, e.g., a selection marker or a reporter gene.

In one embodiment, the vector comprising a nucleic acid sequence encoding a polypeptide, e.g., a recombinant polypeptide, further comprises a promoter sequence responsible for the recruitment of polymerase to enable transcription initiation for expression of the polypeptide, e.g., the recombinant polypeptide. In one embodiment, promoter sequences suitable for the methods described herein are usually associated with enhancers to drive high amounts of transcription and hence deliver large copies of the target exogenous mRNA. In an embodiment, the promoter comprises cytomegalovirus (CMV) major immediate early promoters (Xia, Bringmann et al. 2006) and the SV40 promoter (Chernajovsky, Mory et al. 1984), both derived from their namesake viruses or promoters derived therefrom. Several other less common viral promoters have been successfully employed to drive transcription upon inclusion in an expression vector including Rous Sarcoma virus long terminal repeat (RSV-LTR) and Moloney murine leukaemia virus (MoMLV) LTR (Papadakis, Nicklin et al. 2004). In another embodiment, specific endogenous mammalian promoters can be utilized to drive constitutive transcription of a gene of interest (Pontiller, Gross et al. 2008). The CHO specific Chinese Hamster elongation factor 1-alpha (CHEF1α) promoter has provided a high yielding alternative to viral based sequences (Deer, Allison 2004). In addition to promoters, the vectors described herein further comprise an enhancer region as described above; a specific nucleotide motif region, proximal to the core promoter, which can recruit transcription factors to upregulate the rate of transcription (Riethoven 2010). Similar to promoter sequences, these regions are often derived from viruses and are encompassed within the promoter sequence such as hCMV and SV40 enhancer sequences, or may be additionally included such as adenovirus derived sequences (Gaillet, Gilbert et al. 2007).

In one embodiment, the vector comprising a nucleic acid sequence encoding a product, e.g., a polypeptide, e.g, a recombinant polypeptide, described herein further comprises a nucleic acid sequence that encodes a selection marker. In one embodiment, the selectable marker comprises glutamine synthetase (GS); dihydrofolate reductase (DHFR) e.g., an enzyme which confers resistance to methotrexate (MTX); or an antibiotic marker, e.g., an enzyme that confers resistance to an antibiotic such as: hygromycin, neomycin (G418), zeocin, puromycin, or blasticidin. In another embodiment, the selection marker comprises or is compatible with the Selexis selection system (e.g., SUREtechnology Platform™ and Selexis Genetic Elements™ commercially available from Selexis SA) or the Catalant selection system.

In one embodiment, the vector comprising a nucleic acid sequence encoding a recombinant product described herein comprises a selection marker that is useful in identifying a cell or cells comprise the nucleic acid encoding a recombinant product described herein. In another embodiment, the selection marker is useful in identifying a cell or cells that comprise the integration of the nucleic acid sequence encoding the recombinant product into the genome, as described herein. The identification of a cell or cells that have integrated the nucleic acid sequence encoding the recombinant protein can be useful for the selection and engineering of a cell or cell line that stably expresses the product.

Suitable vectors for use are commercially available, and include vectors associated with the GS Expression System™, GS Xceed™ Gene Expression System, or Potelligent® CHOK1SV technology available from Lonza Biologics, Inc, e.g., vectors as described in Fan et al., Pharm. Bioprocess. (2013); 1(5):487-502, which is incorporated herein by reference in its entirety. GS expression vectors comprise the GS gene, or a functional fragment thereof (e.g., a GS mini-gene), and one or more, e.g., 1, 2, or 3, or more, highly efficient transcription cassettes for expression of the gene of interest, e.g., a nucleic acid encoding a recombinant polypeptide described herein. A GS mini-gene comprises, e.g., consists of, intron 6 of the genomic CHO GS gene. In one embodiment, a GS vector comprises a GS gene operably linked to a SV40L promoter and one or two polyA signals. In another embodiment, a GS vector comprises a GS gene operably linked to a SV40E promoter, SV40 splicing and polyadenylation signals. In such embodiments, the transcription cassette, e.g., for expression of the gene of interest or recombinant polypeptide described herein, includes the hCMV-MIE promoter and 5′ untranslated sequences from the hCMV-MIE gene including the first intron. Other vectors can be constructed based on GS expression vectors, e.g., wherein other selection markers are substituted for the GS gene in the expression vectors described herein.

Vectors suitable for use in the methods described herein include, but are not limited to, other commercially available vectors, such as, pcDNA3.1/Zeo, pcDNA3.1/CAT, pcDNA3.3TOPO (Thermo Fisher, previously Invitrogen); pTarget, HaloTag (Promega); pUC57 (GenScript); pFLAG-CMV (Sigma-Aldrich); pCMV6 (Origene); pEE12 or pEE14 (Lonza Biologics), or pBK-CMV/pCMV-3Tag-7/pCMV-Tag2B (Stratagene).

### Cells and Cell Culture

In embodiments, the cell is a mammalian cell. In other embodiments, the cell is a cell other than a mammalian cell. In an embodiment, the cell is a mouse, rat, Chinese hamster, Syrian hamster, monkey, ape, dog, horse, ferret, or cat. In embodiments, the cell is a mammalian cell, e.g., a human cell or a rodent cell, e.g., a hamster cell, a mouse cell, or a rat cell. In another embodiment, the cell is from a duck, parrot, fish, insect, plant, fungus, or yeast. In one embodiment, the cell is an Archaebacteria. In an embodiment, the cell is a species of Actinobacteria, e.g., Mycobacterium tuberculosis).

In one embodiment, the cell is a Chinese hamster ovary (CHO) cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1SV GS knockout cell (Lonza Biologics, Inc.). The CHO FUT8 knockout cell is, for example, the Potelligent® CHOK1 SV (Lonza Biologics, Inc.).

In another embodiment, the cell is a HeIa, HEK293, HT1080, H9, HepG2, MCF7, Jurkat, NIH3T3, PC12, PER.C6, BHK (baby hamster kidney cell), VERO, SP2/0, NS0, YB2/0, Y0, EB66, C127, L cell, COS, e.g., COS1 and COS7, QC1-3, CHOK1, CHOK1SV, Potelligent CHOK1SV, CHO GS knockout, CHOK1SV GS-KO, CHOS, CHO DG44, CHO DXB11, and CHOZN, or any cells derived therefrom. In one embodiment, the cell is a stem cell. In one embodiment, the cell is a differentiated form of any of the cells described herein. In one embodiment, the cell is a cell derived from any primary cell in culture.

In an embodiment, the cell is any one of the cells described herein that comprises an exogenous nucleic acid encoding a recombinant polypeptide, e.g., expresses a recombinant polypeptide, e.g., a recombinant polypeptide selected from Table 1 or 2.

### Large Scale Production

The methods described herein are of use in analyzing samples, e.g., samples produced by devices, facilities and methods of manufacturing and production. The devices, facilities, and methods of manufacturing and production described herein are suitable for culturing any desired cell line including prokaryotic and/or eukaryotic cell lines. Further, in embodiments, the devices, facilities and methods of manufacturing and production are suitable for culturing suspension cells or anchorage-dependent (adherent) cells and are suitable for production operations configured for production of pharmaceutical and biopharmaceutical products—such as polypeptide products, nucleic acid products (for example DNA or RNA), or cells and/or viruses such as those used in cellular and/or viral therapies.

In embodiments, the cells express or produce a product, such as a recombinant therapeutic or diagnostic product. Examples of products produced by cells include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in embodiments, methods described herein are of use in analyzing samples, e.g., samples produced by devices, facilities and methods of manufacturing and production. The devices, facilities and methods of manufacturing and production allow for the production of eukaryotic cells, e.g., mammalian cells or lower eukaryotic cells such as for example yeast cells or filamentous fungi cells, or prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesised by the eukaryotic cells in a large-scale manner. Unless stated otherwise herein, the devices, facilities, and methods can include any desired volume or production capacity including but not limited to bench-scale, pilot-scale, and full production scale capacities.

In embodiments, devices, facilities, and methods of manufacturing and production allow for the production of cells and products of the cells, especially proteins, peptides (discussed in detail above), antibiotics or amino acids, synthesized by cells, e.g., mammalian cells, in a large-scale manner.

A wide array of flasks, bottles, reactors, and controllers allow the production and scale up of cell culture systems. The system can be chosen based, at least in part, upon its correlation with a desired glycan property or properties. Cells can be grown, for example, as batch, fed-batch, perfusion, or continuous cultures. Production parameters that can be selected include, e.g., addition or removal of media including when (early, middle or late during culture time) and how often media is harvested; increasing or decreasing speed at which cell cultures are agitated; increasing or decreasing temperature at which cells are cultured; adding or removing media such that culture density is adjusted; selecting a time at which cell cultures are started or stopped; and selecting a time at which cell culture parameters are changed. Such parameters can be selected for any of the batch, fed-batch, perfusion and continuous culture conditions.

In embodiments, the cultivated cells for large scale production are eukaryotic cells, e.g., animal cells, e.g., mammalian cells. The mammalian cells can be, for example, human cell lines, mouse myeloma (NSO)-cell lines, Chinese hamster ovary (CHO)-cell lines or hybri-doma-cell lines. Preferably the mammalian cells are CHO-cell lines.

In embodiments, the cultivated cells for large scale production are used to produce antibodies discussed in detail above, e.g., monoclonal antibodies, and/or recombinant proteins, e.g., recombinant proteins for therapeutic use. In embodiments, the cells produce peptides, amino acids, fatty acids or other useful biochemical intermediates or metabolites.

In embodiments, the cells for large scale production are eukaryotic cells, biochemical markers, recombinant peptides or nucleotide sequences of interest, proteins, yeast, insect cells, stable or viral infected, avian cells or mammalian cells such as CHO cells, monkey cells, lytic products and the like for medical, research or commercial purposes.

In embodiments, the cells for large scale production are prokaryotic cells, strains of Gram-positive cells such as Bacillus and Streptomyces. In embodiments, the host cell is of phylum Firmicutes, e.g., the host cell is Bacillus. Bacillus that can be used are, e.g. the strains B. subtilis, B. amyloliquefaciens, B. licheniformis, B. natto, B. megaterium, etc. In embodiments, the host cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus Ohio 43210-1214.

In embodiments, the prokaryotic cells for large scale production are Gram negative cells, such as Salmonella spp. or E. coli, e.g., the strains TG1, W3110, DH1, XL1-Blue and Origami, which are commercially available.

Suitable host cells are commercially available, for example, from culture collections such as the DSMZ (Deutsche Sammlung von Mikroorganismen and Zellkulturen GmbH, Braunschweig, Germany).

In an embodiment, the cell culture is carried out as a batch culture, fed-batch culture, draw and fill culture, or a continuous culture. In an embodiment, the cell culture is a suspension culture. In one embodiment, the cell or cell culture is placed in vivo for expression of the recombinant polypeptide, e.g., placed in a model organism or a human subject.

In one embodiment, the culture media is free of serum. Serum-free and protein-free media are commercially available, e.g., Lonza Biologics.

Suitable media and culture methods for mammalian cell lines are well-known in the art, as described in U.S. Pat. No. 5,633,162, for instance. Examples of standard cell culture media for laboratory flask or low density cell culture and being adapted to the needs of particular cell types are for instance: Roswell Park Memorial Institute (RPMI) 1640 medium (Morre, G., The Journal of the American Medical Association, 199, p. 519 f. 1967), L-15 medium (Leibovitz, A. et al., Amer. J. of Hygiene, 78, 1p. 173 ff, 1963), Dulbecco's modified Eagle's medium (DMEM), Eagle's minimal essential medium (MEM), Ham's F12 medium (Ham, R. et al., Proc. Natl. Acad. Sc. 53, p288 ff. 1965) or Iscoves' modified DMEM lacking albumin, transferrin and lecithin (Iscoves et al., J. Exp. med. 1, p. 923 ff., 1978). For instance, Ham's F10 or F12 media were specially designed for CHO cell culture. Other media specially adapted to CHO cell culture are described in EP-481 791. It is known that such culture media can be supplemented with fetal bovine serum (FBS, also called fetal calf serum FCS), the latter providing a natural source of a plethora of hormones and growth factors. The cell culture of mammalian cells is nowadays a routine operation well-described in scientific textbooks and manuals, it is covered in detail e.g. in R. Ian Fresney, Culture of Animal cells, a manual, 4^{th} edition, Wiley-Liss/N.Y., 2000.

Other suitable cultivation methods are known to the skilled artisan and may depend upon the recombinant polypeptide product and the host cell utilized. It is within the skill of an ordinarily skilled artisan to determine or optimize conditions suitable for the expression and production of the recombinant polypeptide to be expressed by the cell.

In one aspect, the cell or cell line for large scale production comprises an exogenous nucleic acid that encodes a product, e.g., a recombinant polypeptide. In an embodiment, the cell or cell line expresses the product, e.g., a therapeutic or diagnostic product. Methods for genetically modifying or engineering a cell to express a desired polypeptide or protein are well known in the art, and include, for example, transfection, transduction (e.g., viral transduction), or electroporation.

Physical methods for introducing a nucleic acid, e.g., an exogenous nucleic acid or vector described herein, into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY).

Chemical means for introducing a nucleic acid, e.g., an exogenous nucleic acid or vector described herein, into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In embodiments, the integration of the exogenous nucleic acid into a nucleic acid of the host cell, e.g., the genome or chromosomal nucleic acid of the host cell is desired. Methods for determining whether integration of an exogenous nucleic acid into the genome of the host cell has occurred can include a GS/MSX selection method. The GS/MSX selection method uses complementation of a glutamine auxotrophy by a recombinant GS gene to select for high-level expression of proteins from cells. Briefly, the GS/MSX selection method comprises inclusion of a nucleic acid encoding glutamine synthetase on the vector comprising the exogenous nucleic acid encoding the recombinant polypeptide product. Administration of methionine sulfoximine (MSX) selects cells that have stably integrated into the genome the exogenous nucleic acid encoding both the recombinant polypeptide and GS. As GS can be endogenously expressed by some host cells, e.g., CHO cells, the concentration and duration of selection with MSX can be optimized to identify high producing cells with stable integration of the exogenous nucleic acid encoding the recombinant polypeptide product into the host genome. The GS selection and systems thereof is further described in Fan et al., Pharm. Bioprocess. (2013); 1(5):487-502, which is incorporated herein by reference in its entirety.

Other methods for identifying and selecting cells that have stably integrated the exogenous nucleic acid into the host cell genome can include, but are not limited to, inclusion of a reporter gene on the exogenous nucleic acid and assessment of the presence of the reporter gene in the cell, and PCR analysis and detection of the exogenous nucleic acid. In one embodiment, the cells selected, identified, or generated using the methods described herein are capable of producing higher yields of protein product than cells that are selected using only a selection method for the stable expression, e.g., integration of exogenous nucleic acid encoding the recombinant polypeptide. In an embodiment, the cells selected, identified, or generated using the methods described herein produce 2-fold, 3-fold, 4-fold, 5-fold, 6-fold, 7-fold, 8-fold, 9-fold, or 10-fold or more of the product, e.g., recombinant polypeptide, as compared to cells that were not contacted with an inhibitor of protein degradation, or cells that were only selected for stable expression, e.g., integration, of the exogenous nucleic acid encoding the recombinant polypeptide.

### Methods for Cell Line and Recombinant Polypeptide Production

Methods for recovering and purification of a product, e.g., a recombinant polypeptide, are well established in the art. For recovering the recombinant polypeptide product, a physical or chemical or physical-chemical method is used. The physical or chemical or physical-chemical method can be a filtering method, a centrifugation method, an ultracentrifugation method, an extraction method, a lyophilization method, a precipitation method, a crystallization method, a chromatography method or a combination of two or more methods thereof. In an embodiment, the chromatography method comprises one or more of size-exclusion chromatography (or gel filtration), ion exchange chromatography, e.g., anion or cation exchange chromatography, affinity chromatography, hydrophobic interaction chromatography, and/or multimodal chromatography.

The methods described herein are suitable for analyzing samples produced by manufacturing and production methods that culture any desired cell including prokaryotic cells and/or eukaryotic cells. The methods of manufacturing and production can be performed in, e.g., a reactor, e.g., a bioreactor. Further, in embodiments, samples and products can be produced using devices, facilities and production methods suitable for culturing suspension cells or anchorage-dependent (adherent) cells and suitable for production operations configured for production of molecular products—such as polypeptide products—or cells and/or viruses such as those used in cellular and/or viral therapies.

In embodiments, the cells express or produce a product, such as a recombinant therapeutic or diagnostic product. As described in more detail below, examples of products produced by cells include, but are not limited to, antibody molecules (e.g., monoclonal antibodies, bispecific antibodies), fusion proteins (e.g., Fc fusion proteins, chimeric cytokines), other recombinant proteins (e.g., glycosylated proteins, enzymes, hormones), or lipid-encapsulated particles (e.g., exosomes, virus-like particles). In embodiments, the devices, facilities and methods can be used for producing biosimilars.

In embodiments, devices, facilities and production methods allow for the production of eukaryotic cells, e.g., mammalian cells, and/or products of the eukaryotic cells, e.g., proteins, peptides, antibiotics or amino acids, synthesized by the eukaryotic cells in a large-scale manner. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and production methods can include any suitable reactor(s) including but not limited to stirred tank, airlift, fiber, microfiber, hollow fiber, ceramic matrix, fluidized bed, fixed bed, spouted bed, and/or stirred tank bioreactors. For example, in some aspects, an example bioreactor unit can perform one or more, or all, of the following: feeding of nutrients and/or carbon sources, injection of suitable gas (e.g., oxygen), flow of fermentation or cell culture medium, separation of gas and liquid phases, maintenance of temperature, maintenance of pH level, agitation (e.g., stirring), and/or cleaning/sterilizing. Example reactor units, such as a fermentation unit, may contain 1, 2, 3, 4, 5, 10, 15, 20, 25, 30, 35, 40, 45, 50, 60, 70, 80, 90, or 100, or more bioreactors. In various embodiments, the bioreactor can be suitable for batch, semi fed-batch, fed-batch, perfusion, and/or continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass. In some embodiments, suitable reactors can be round, e.g., cylindrical. In some embodiments, suitable reactors can be square, e.g., rectangular. Square reactors may in some cases provide benefits over round reactors such as ease of use (e.g., loading and setup by skilled persons), greater mixing and homogeneity of reactor contents, and lower floor footprint.

In embodiments and unless stated otherwise herein, the devices, facilities, and production methods described herein can also include any suitable unit operation and/or equipment not otherwise mentioned, such as operations and/or equipment for separation, purification, and isolation of such products. Any suitable facility and environment can be used, such as traditional stick-built facilities, modular facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

In embodiments, the cells are eukaryotic cells, e.g., mammalian cells. The mammalian cells can be for example human or rodent or bovine cell lines or cell strains. Examples of such cells, cell lines or cell strains are e.g. mouse myeloma (NS0)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EBl, EB2, EB3, oncolytic or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBvl3.

In one embodiment, the eukaryotic cells are stem cells. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In embodiments, the cultivated cells are eukaryotic cells, e.g., mammalian cells. The mammalian cells can be for example human cell lines, mouse myeloma (NSO)-cell lines, Chinese hamster ovary (CHO)-cell lines or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.).

In embodiments, the cell is a yeast cell (e.g., S. cerevisae, T. reesei), an insect cell (e.g., Sf9), an algae cell (e.g., cyanobacteria), or a plant cell (e.g., tobacco, alfalfa, Physcomitrella patens). In one embodiment, the cell is a rodent cell. In another embodiment, the cell is a HeLa, HEK293, HT1080, H9, HepG2, MCF7, Jurkat, NIH3T3, PC12, PER.C6, BHK (baby hamster kidney cell), VERO, SP2/0, NS0, YB2/0, Y0, EB66, C127, L cell, COS, e.g., COS1 and COS7, QC1-3, CHO-K1.

In embodiments, the cell is a stem cell. In one embodiment, the cell is a differentiated form of any of the cells described herein. In one embodiment, the cell is a cell derived from any primary cell in culture.

In embodiments, the cell is a hepatocyte such as a human hepatocyte, animal hepatocyte, or a non-parenchymal cell. For example, the cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabit hepatocytes (including New Zealand White hepatocytes). Example hepatocytes are commercially available from Triangle Research Labs, LLC, 6 Davis Drive Research Triangle Park, N.C., USA 27709.

In one embodiment, the eukaryotic cell is a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisae, cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii,), the Geotrichum genus (e.g. Geotrichum fermentans), Hansenula polymorpha, Yarrowia lipolytica, or Schizosaccharomyces pombe. Preferred is the species Pichia pastoris. Examples for Pichia pastoris strains are X33, GS115, KM71, KM71H; and CBS7435.

In one embodiment, the eukaryotic cell is a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

In one embodiment, the eukaryotic cell is an insect cell (e.g., Sf9, Mimic™ Sf9, f21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one embodiment, the cell is a bacterial or prokaryotic cell.

In embodiments, the prokaryotic cell is a Gram-positive cells such as Bacillus, Streptomyces Streptococcus, Staphylococcus or Lactobacillus. Bacillus that can be used is, e.g. the B. subtilis, B. amyloliquefaciens, B. licheniformis, B. natto, or B. megaterium. In embodiments, the cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus Ohio 43210-1214.

In one embodiment, the prokaryotic cell is a Gram-negative cell, such as Salmonella spp. or Escherichia coli, such as e.g., TG1, TG2, W3110, DH1, DHB4, DH5a, HMS 174, HMS174 (DE3), NM533, C600, HB101, JM109, MC4100, XL1-Blue and Origami, as well as those derived from E. coli B-strains, such as for example BL-21 or BL21 (DE3), all of which are commercially available.

Suitable host cells are commercially available, for example, from culture collections such as the DSMZ (Deutsche Sammlung von Mikroorganismen and Zellkulturen GmbH, Braunschweig, Germany) or the American Type Culture Collection (ATCC).

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

### NUMBERED EMBODIMENTS

1. A method of separating a compound of Formula I, e.g., tropolone, from another component of a sample comprising:

contacting the sample with a partially or fully fluorinated alkyl or aryl, e.g., a fluorophenyl, e.g., a pentafluorophenylpropyl, moiety, under conditions wherein the compound of Formula I, e.g., tropolone, associates with, e.g., binds to or is retained by, the moiety to a greater extent than the component,

thereby separating the compound of Formula I, e.g., tropolone, from the component, wherein Formula I is:

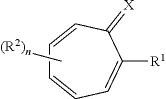

and wherein:

X is O or S;

R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;

each R² is independently C₁-C₆ alkyl, C₁-C₆ heteroalkyl, N(R^{4a})(R^{4b}), C(O)N(R^{4a})(R^{4b}), or N(R^{4a})C(O)R5; or

two R² are joined to form a heterocyclyl ring optionally substituted with one or more R⁶; or R¹ and R² are joined to form a heterocyclyl ring optionally substituted with one or more R⁶;

R³ is hydrogen, C₁-C₆ alkyl, or C₁-C₆ heteroalkyl;

R^{4a} and R^{4b} are independently hydrogen, C₁-C₆ alkyl, or C₁-C₆ heteroalkyl;

R⁵ is C₁-C₆ alkyl or C₁-C₆ heteroalkyl;

each R⁶ is independently C₁-C₆ alkyl, C₁-C₆ heteroalkyl, halo, oxo, or cyano; and

n is 0, 1, 2, 4, or 5.

2. The method of paragraph 1, wherein the moiety comprises a pentafluorophenylpropyl group.
3. The method of either of paragraphs 1 or 2, wherein the pentafluorophenylpropyl group is associated with, e.g., bound to, e.g., covalently bound to, a substrate.
4. The method of paragraph 3, wherein the substrate comprises an insoluble substrate, e.g., a chromatography matrix, e.g., a silica gel.
5. The method of any of paragraphs 1-4, comprising contacting the moiety with one or more mobile phases (e.g., one or two mobile phases) under conditions wherein the compound is preferentially eluted.
6. The method of any of paragraphs 1-5, wherein the method comprises subjecting the sample to a liquid chromatography (LC) separation.
7. A method of evaluating the presence, e.g., the level, of a compound of Formula I, e.g., tropolone, in a sample comprising a product, comprising:

a) i) providing an aliquot of a sample, e.g., a compound of Formula I (e.g., tropolone) depleted phase, e.g., a mobile phase, wherein the compound of Formula I, e.g., tropolone, has been separated from another component of the sample, or
ii) subjecting the sample to conditions wherein the compound of Formula I, e.g., tropolone, is separated from another component of the sample, e.g., to form a compound of Formula I, e.g., tropolone, enriched phase or aliquot and a compound of Formula I, e.g., tropolone, depleted phase or aliquot; and

b) evaluating the presence, e.g., the level, of the compound of Formula I, e.g., tropolone, e.g., determining a value for the level of the compound of Formula I, e.g., tropolone, in the sample:
i) using tandem mass spectrometry (MS²), or
ii) using ultraviolet (UV) absorption, e.g., UV absorption at about 242 nm or about 238 nm,
   thereby analyzing the sample,

wherein Formula I is:

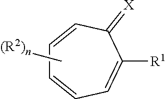

and wherein:

X is O or S;

R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;

each R² is independently C₁-C₆ alkyl, C₁-C₆ heteroalkyl, N(R^{4a})(R^{4b}), C(O)N(R^{4a})(R^{4b}), or N(R^{4a})C(O)R5; or

two R² are joined to form a heterocyclyl ring optionally substituted with one or more R⁶; or R¹ and R² are joined to form a heterocyclyl ring optionally substituted with one or more R⁶;

R³ is hydrogen, C₁-C₆ alkyl, or C₁-C₆ heteroalkyl;

R^{4a} and R^{4b} are independently hydrogen, C₁-C₆ alkyl, or C₁-C₆ heteroalkyl;

R⁵ is C₁-C₆ alkyl or C₁-C₆ heteroalkyl;

each R⁶ is independently C₁-C₆ alkyl, C₁-C₆ heteroalkyl, halo, oxo, or cyano; and

n is 0, 1, 2, 4, or 5.

8. The method of paragraph 7, wherein a) comprises providing an aliquot of a sample, e.g., a compound of Formula I, e.g., tropolone, depleted phase, e.g., a mobile phase, wherein the compound of Formula I, e.g., tropolone, has been separated from another component of the sample.
9. The method of paragraph 7, wherein a) comprises subjecting the sample to conditions wherein the compound of Formula I, e.g., tropolone, is separated from another component of the sample, e.g., to form a compound of Formula I, e.g., tropolone, enriched phase or aliquot and a compound of Formula I, e.g., tropolone, depleted phase or aliquot.
10. The method of any of any of paragraphs 7-9, wherein a) comprises subjecting the sample to a liquid chromatography (LC) separation.
11. The method of any of paragraphs 7-10, wherein a) comprises contacting the sample with a partially or fully fluorinated alkyl or aryl, e.g., a fluorophenyl, e.g., a pentafluorophenylpropyl, moiety, under conditions wherein the compound of Formula I, e.g., tropolone, associates with, e.g., binds to, or is retained by, the moiety to a greater extent than the component.
12. The method of paragraph 11, wherein the moiety comprises a pentafluorophenylpropyl group.
13. The method of any of paragraphs 7-12, wherein b) comprises comprising evaluating the level or presence of the compound of Formula I, e.g., tropolone, e.g., determining a value for the level of the compound of Formula I, e.g., tropolone, in the sample using tandem mass spectrometry (MS²).
14. The method of any of paragraphs 7-12, wherein b) comprises evaluating the level or presence of the compound of Formula I, e.g., tropolone, e.g., determining a value for the level of the compound of Formula I, e.g., tropolone, in the sample using ultraviolet (UV) absorption, e.g., UV absorption at about 242 nm or about 238 nm.
15. The method of any of paragraphs 7, 11, or 12 comprising: a)i) and b)i).
16. The method of any of paragraphs 7, 11, or 12 comprising: a)i) and b)ii).
17. The method of any of paragraphs 7, 11, or 12 comprising: a)ii) and b)i).
18. The method of any of paragraphs 7, 11, or 12 comprising: a)ii) and b)ii).
19. The method of any of paragraphs 7-18, wherein the linear range of the method with regard to determining a value for the level of the compound of Formula I, e.g., tropolone, present in the sample is about 0.1-10000, 0.2-8000, 0.3-7000, 0.4-6000, 0.5-5000, 0.5-4000, 0.5-3000, 0.5-2000, or 0.5-1000 μg/ml, e.g., 0.5-1000 μg/ml.
20. The method of any of paragraphs 7-19, wherein the lower limit of the linear range of the method with regard to determining a value for the level of the compound of Formula I, e.g., tropolone, in the sample is about 0.01, 0.05, 0.1, 0.2, 0.3, 0.35, 0.4, 0.45, 0.5, 0.6, 0.7, 0.8, 0.9, or 1 μg/ml, e.g., 0.5 μg/ml.
21. The method of any of paragraphs 7-20, wherein the upper limit of the linear range of the method with regard to determining a value for the level of the compound of Formula I, e.g., tropolone, in the sample is about 500, 600, 700, 800, 900, 1000, 1200, 1400, 1600, 1800, 2000, 3000, 4000, 5000, 6000, 7000, 8000, 9000, or 10,000 μg/ml, e.g., 1000 μg/ml.
22. The method of any of paragraphs 7-21, wherein the precision (e.g., represented by the standard deviation between replicate samples) of the method with regard to determining a value for the level of the compound of Formula I, e.g., tropolone, present in the sample can be less than or equal to about 50, 40, 30, 25, 20, 19, 18, 17, 16, 15, 14, 13, 12, 11, 10, 9, 8, 7, 6, 5, 4, 3, 2, or 1%, e.g., 17, 16.5, or 16%.
23. The method of any of paragraphs 7-22, wherein the accuracy (e.g., represented by average single point spike recovery in three different samples) of the method with regard to determining a value for the level of the compound of Formula I, e.g., tropolone, present in the sample is greater than or equal to about 70, 75, 80, 81, 82, 83, 84, 85, 86, 87, 88, 89, 90, 91, 92, 93, 94, or 95%, e.g., 91%.
24. The method of any of paragraphs 7-23, wherein the lower limit of detection of the method with regard to determining a value for the level of the compound of Formula I, e.g., tropolone, present in the sample is about 1, 1.5, 2, 2.5, 3, 3.5, 4, 4.5, 5, 5.5, 6, 6.5, 7, 7.5, 8, 8.5, 9, 9.5, or 10 μg/ml, e.g., 5 μg/ml.
25. The method of either paragraph 6 or 10, wherein the LC is reversed phase chromatography.
26. The method of either paragraph 6 or 10, wherein the LC is not reversed phase chromatography.
27. The method of either paragraph 6 or 10, wherein the LC comprises using a stationary phase comprising a partially or fully fluorinated alkyl or aryl, e.g., a fluorophenyl, e.g., a pentafluorophenylpropyl, group.
28. The method of paragraph 27, wherein the LC comprises using a stationary phase comprising a fluorophenyl group.
29. The method of paragraph 27, wherein the LC comprises using a stationary phase comprising a pentafluorophenylpropyl group.
30. The method of any of paragraphs 6, 10, or 25-29, wherein the LC comprises using a first mobile phase and a second mobile phase.
31. The method of paragraph 30, wherein the first mobile phase comprises formic acid in water, e.g., about 0.01%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, or 1% formic acid in water.
32. The method of paragraph 31, wherein the first mobile phase comprises about 0.1% formic acid in water.
33. The method of paragraph 30, wherein the second mobile phase comprises formic acid in acetonitrile, e.g., about 0.01%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, or 1% formic acid in acetonitrile.
34. The method of paragraph 33, wherein the second mobile phase comprises about 0.1% formic acid in acetonitrile.
35. The method of either of paragraphs 33 or 34, wherein the second mobile phase comprises at least about 50, 55, 60, 65, 70, 75, 80, 85, 90, 95, or 100% acetonitrile, e.g., about 100% acetonitrile.
36. The method of any of paragraphs 6, 10, or 25-35, wherein the LC comprises: using a stationary phase comprising a pentafluorophenylpropyl group, and using a first mobile phase and a second mobile phase, wherein the first mobile phase comprises about 0.1% formic acid in water, and wherein the second mobile phase comprises about 0.1% formic acid in acetonitrile.
37. The method of any of paragraphs 6, 10, or 25-36, wherein the LC comprises using a Discovery HS F5-3 column.
38. The method of any of paragraphs 7-13, 15, 17, and 19-37, wherein using MS² comprises selected reaction monitoring (SRM).
39. The method of any of paragraphs 7-13, 15, 17, and 19-37, wherein using MS² comprises multiple reaction monitoring (MRM), e.g., parallel reaction monitoring (PRM).
40. The method of either of paragraphs 38 or 39, wherein SRM or MRM (e.g., PRM), is used to monitor one or more transitions selected from transition i, ii, iii, iv, v, and vi of Table 1.
41. The method of paragraph 40, wherein SRM or MRM (e.g., PRM), is used to monitor transition i.
42. The method of paragraph 40, wherein SRM or MRM (e.g., PRM), is used to monitor transition ii.
43. The method of paragraph 40, wherein SRM or MRM (e.g., PRM), is used to monitor transition iii.
44. The method of paragraph 40, wherein SRM or MRM (e.g., PRM), is used to monitor transition iv.
45. The method of paragraph 40, wherein SRM or MRM (e.g., PRM), is used to monitor transition v.
46. The method of paragraph 40, wherein SRM or MRM (e.g., PRM), is used to monitor transition vi.
47. A reaction mixture comprising a partially or fully fluorinated alkyl or aryl, e.g., a fluorophenyl, e.g., a pentafluorophenylpropyl, moiety, and a sample comprising a compound of Formula I, e.g., tropolone, another component, and optionally a product, wherein Formula I is given by:

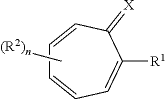

and wherein:

X is O or S;

R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;

each R² is independently C₁-C₆ alkyl, C₁-C₆ heteroalkyl, N(R^{4a})(R^{4b}), C(O)N(R^{4a})(R^{4b}), or N(R^{4a})C(O)R5; or

two R² are joined to form a heterocyclyl ring optionally substituted with one or more R⁶; or R¹ and R² are joined to form a heterocyclyl ring optionally substituted with one or more R⁶;

R³ is hydrogen, C₁-C₆ alkyl, or C₁-C₆ heteroalkyl;

R^{4a} and R^{4b} are independently hydrogen, C₁-C₆ alkyl, or C₁-C₆ heteroalkyl;

R⁵ is C₁-C₆ alkyl or C₁-C₆ heteroalkyl;

each R⁶ is independently C₁-C₆ alkyl, C₁-C₆ heteroalkyl, halo, oxo, or cyano; and

n is 0, 1, 2, 4, or 5.

48. A method of manufacturing a product, e.g., a recombinant polypeptide, comprising providing a sample comprising the product and optionally a compound of Formula I, e.g., tropolone, wherein:

the sample is analyzed by a method of any of paragraphs 7-43, 45, or 46, or

the compound of Formula I, e.g., tropolone, is separated from another component of the sample by a method of any of paragraphs 1-6,

wherein Formula I is given by:

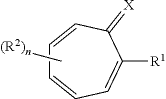

and wherein:

X is O or S;

R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;

each R² is independently C₁-C₆ alkyl, C₁-C₆ heteroalkyl, N(R^{4a})(R^{4b}), C(O)N(R^{4a})(R^{4b}), or N(R^{4a})C(O)R5; or

two R² are joined to form a heterocyclyl ring optionally substituted with one or more R⁶; or R¹ and R² are joined to form a heterocyclyl ring optionally substituted with one or more R⁶;

R³ is hydrogen, C₁-C₆ alkyl, or C₁-C₆ heteroalkyl;

R^{4a} and R^{4b} are independently hydrogen, C₁-C₆ alkyl, or C₁-C₆ heteroalkyl;

R⁵ is C₁-C₆ alkyl or C₁-C₆ heteroalkyl;

each R⁶ is independently C₁-C₆ alkyl, C₁-C₆ heteroalkyl, halo, oxo, or cyano; and

n is 0, 1, 2, 4, or 5.

49. The method of paragraph 48, wherein the method of manufacturing comprises expression and secretion from a plurality of cells (e.g., a plurality of CHO cell, e.g., a plurality of GS-CHO cells).
50. The method or reaction mixture of any of paragraphs 1-49, wherein the sample comprises culture supernatant.
51. The method or reaction mixture of any of paragraphs 1-49, wherein the sample comprises cell lysate.
52. The method or reaction mixture of any of paragraphs 1-51, wherein the sample comprises culture supernatant and cell lysate.
53. The method or reaction mixture of any of paragraphs 1-52, wherein the sample was generated by a method of manufacturing a product, e.g., a recombinant polypeptide.
54. The method or reaction mixture of any of paragraphs 1-53, wherein the sample comprises a final product, e.g., a final product formulated for delivery (e.g., administration to a patient).
55. The method or reaction mixture of any of paragraphs 1-54, wherein the product or recombinant polypeptide is a homopolymeric or heteropolymeric polypeptide, e.g., a hormone, growth factor, receptor, antibody, cytokine, receptor ligand, transcription factor or enzyme, preferably an antibody or an antibody fragment, e.g., a human antibody or a humanized antibody or fragment thereof, e.g., a humanized antibody or fragment thereof derived from a mouse, rat, rabbit, goat, sheep, or cow antibody, typically of rabbit origin.
56. The method or reaction mixture of any of paragraphs 1-55, wherein the product or recombinant polypeptide is a therapeutic polypeptide.
57. The method or reaction mixture of any of paragraphs 1-56, wherein the product or recombinant polypeptide is one disclosed in Table 1, Table 2, Table 3, or Table 4.
58. The method or reaction mixture of any of paragraphs 1-57, wherein the product or recombinant polypeptide is an antibody.
59. The method or reaction mixture of paragraph 58, wherein the antibody is a monoclonal antibody.
60. The method or reaction mixture of either of paragraphs 58 or 59, wherein the monoclonal antibody is a therapeutic antibody.
61. The method or reaction mixture of any of paragraphs 49-60, wherein the cells are mammalian cells.
62. The method or reaction mixture of paragraph 61, wherein the cell is a mouse, rat, Chinese hamster, Syrian hamster, monkey, ape, dog, horse, ferret, or cat.
63. The method or reaction mixture of paragraph 61, wherein the cells are Chinese hamster ovary (CHO) cells.
64. The method or reaction mixture of paragraph 63, wherein the CHO cells are CHO-K1 cells, CHO-K1 SV cells, DG44 CHO cells, DUXB11 CHO cells, CHOS cells, CHO GS knock-out cells, CHO FUT8 GS knock-out cells, CHOZN cells, or CHO-derived cells.
65. The method or reaction mixture of paragraph 61, wherein the cells are HeIa, HEK293, HT1080, H9, HepG2, MCF7, Jurkat, NIH3T3, PC12, PER.C6, BHK (baby hamster kidney cell), VERO, SP2/0, NS0, YB2/0, Y0, EB66, C127, L cell, COS, e.g., COS1 and COS7, QC1-3, or any cells derived therefrom.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1: Equipment and Reagents

The following equipment, reagents, and acronyms are used in Examples 2-6.

### Acronyms:

| | |
|---|---|
| BDS | Bulk drug substance |
| LC-MS/MS | Liquid chromatography tandem mass spectrometry |
| LOD | Limit of detection |
| LLOD | Lower limit of detection |
| SRM | Selected reaction monitoring |
| RSD | Relative standard deviation |

### Equipment:

Phenomenex Luna-NH₂ 150×2 mm, 5 μm column, part no. 00F-4378-B0, serial no. H15-228806 and H15-045780.
Supelco Discovery HS F5 150×2.1 mm 3 μm, product no. 567503-U, column no. 149000-03, BL: 8129

### Waters Acquity UPLC System ID 270419.

### Waters Xevo TQ-MS. System ID 270418.

### 5±3° C. Storage: Room G100, Slough.

### Software:

Waters MassLynx™ Mass Spectrometry software

### Waters TargetLynx™ Application Manager

### Materials & Reagents:

Tropolone, Sigma Aldrich, part no. 15702-5G, batch no. BCBR4016V
Eluate 1—formulation: 10 mM sodium phosphate/40 mM sodium chloride, pH 7.5
Eluate 2—formulation: 10 mM sodium phosphate/40 mM sodium chloride/400 mM sodium citrate, pH 6.1
BASM—formulation: 30 mM histidine/histidine HCl, 225 mM sorbitol, pH 6.0

### Example 2: Development of the Method

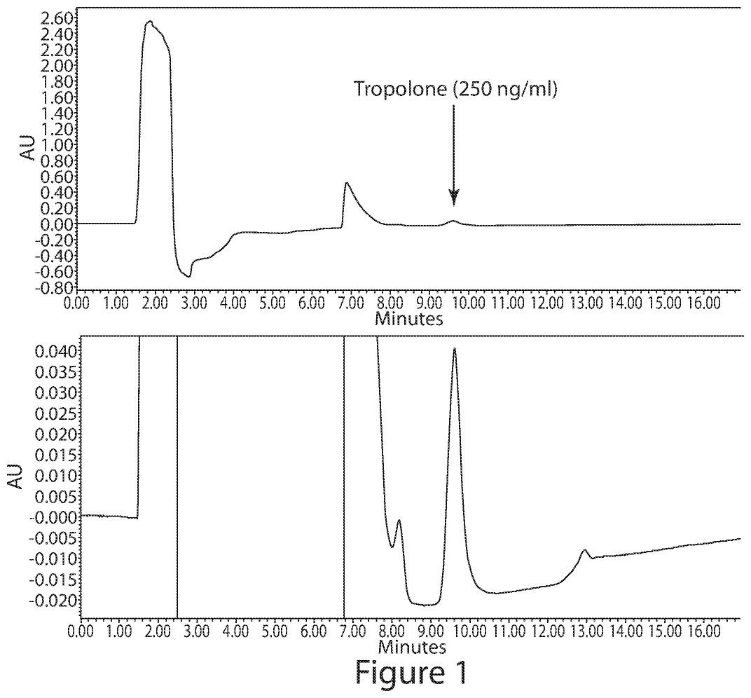

Existing RP-HPLC separation methods and UV detection were used to separate and detect tropolone in a typical sample with typical formulation components. The UV chromatogram shows multiple peaks present, attributable to sample buffer components, and at levels that can make quick and accurate identification and quantification of tropolone difficult (FIG. 1).

Using IntelliStart software, a SRM transition was developed. Tropolone was dissolved in 50% acetonitrile and infused directly into the mass spectrometer with both positive and negative ionisation modes scanned. The results of which are shown in Table 1.

TABLE 1
IntelliStart Developed SRM Transitions

| Transition | Parent | Cone | Daughter | Collision | Ion |
|---|---|---|---|---|---|
| Number | (m/z) | voltage | (m/z) | Energy (V) | Mode |
| i | 123.07 | 8 | 123.07 | 14 | + |
| ii | 123.07 | 8 | 105.00 | 14 | + |
| iii | 123.07 | 8 | 77.00 | 14 | + |
| iv | 123.07 | 8 | 51.00 | 14 | + |
| v | 121.07 | 10 | 121.07 | 4 | − |
| vi | 121.07 | 10 | 65.03 | 4 | − |

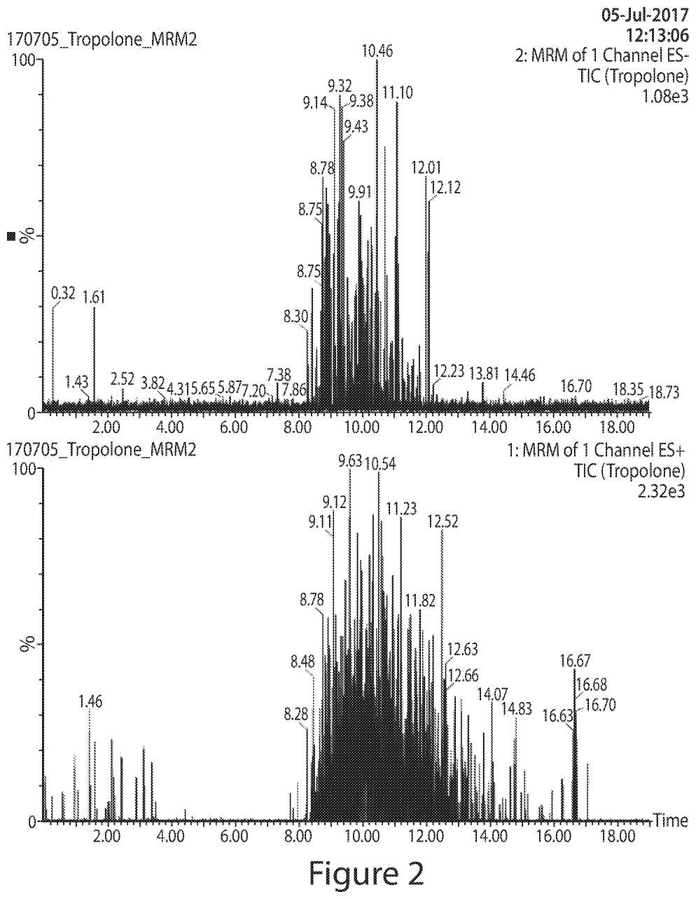

These SRMs were tested for detection of chromatographic separation using a Luna-NH₂ (Phenomenex) LC column using 40 mM ammonium acetate pH 9.45/5% acetonitrile as mobile phase A and acetonitrile as mobile phase B. This configuration showed no analyte retention (FIG. 2), so this column was not used for further experiments.

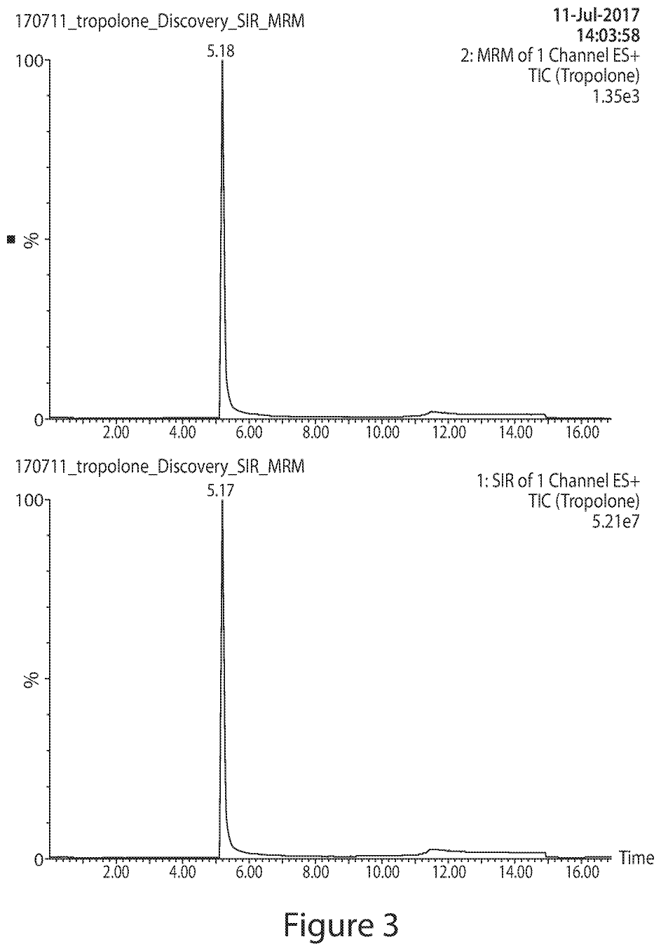

The LC column was switched to a Discovery HS F5-3 (Supelco) using 0.1% formic acid in water as mobile phase A and 0.1% formic acid in acetonitrile as mobile phase B. Using this configuration, both SRMs showed a single, sharp peak (eluting at 5.18 minutes) for an injection of tropolone dissolved in 50:50 mobile phase A:B (FIG. 3).

### Example 3: Method Performance—Linear Rane Precision, and Accuracy

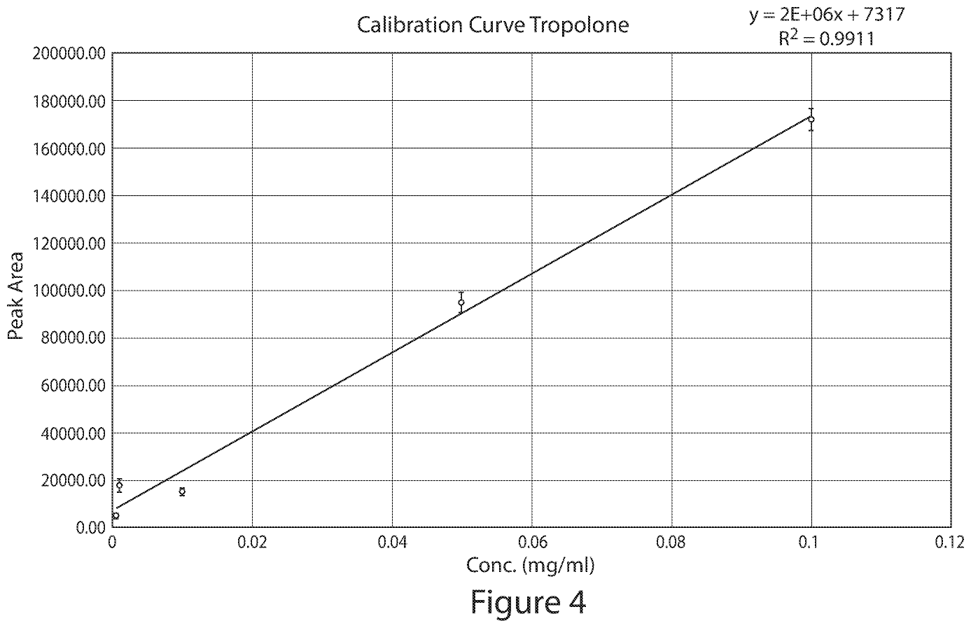

The developed LC-MS method of Example 2 was tested for the following method performance parameters: linear range, accuracy and precision. Linear range was assessed using a 5-point calibration curve (0.5, 1.0, 100.0, 500.0 and 1000.0 μg/mL), analysed across 2 days (1 injection followed by triplicate injection). One further calibration point (0.1 μg/mL) was analysed but found to be below the LOD of the method and so no peak areas were plotted (FIG. 4). Across the 0.5-1000.0 μg/mL range, linearity was found to be R²=0.9911.

Precision was calculated using the average of the relative standard deviation (RSD) from the replicate injections of the standard curve giving a value of 16.56%.

Accuracy was calculated from a single point spike recovery experiment into 3 different process samples giving an average recovery of 91.4%.

### Example 4: Method Performance—Testing in-Process Samples

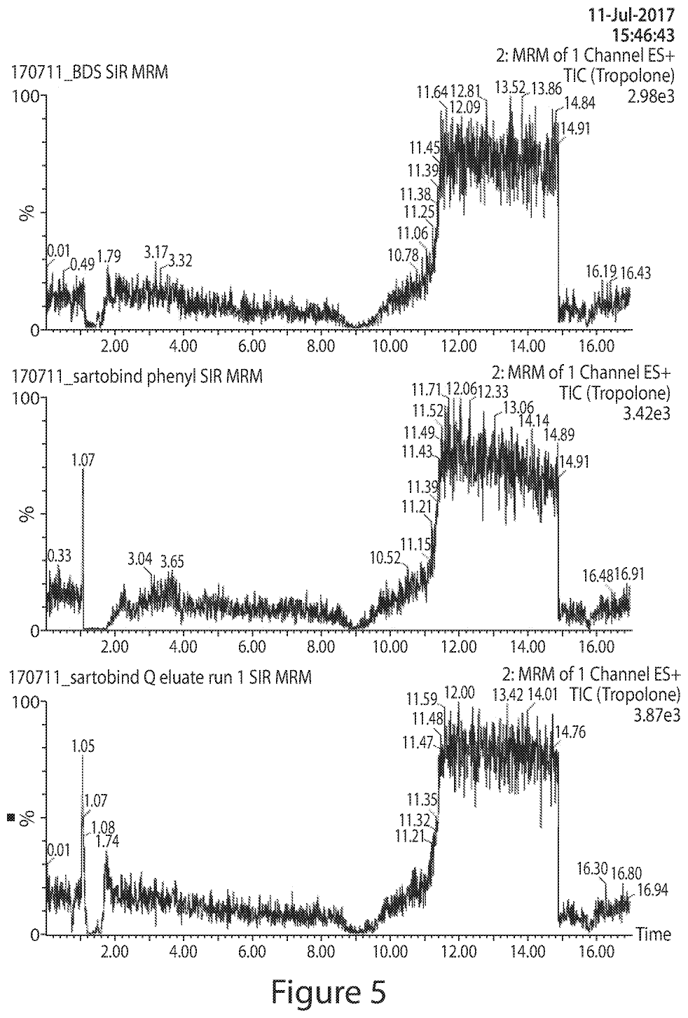

The method developed and tested in Examples 2 and 3 was further tested on three samples from various stages of purification of a manufactured biological product. The three in-process samples tested were from various downstream stages (post Sartobind Q and Sartobind Phenyl columns and bulk drug substance (BDS)) of different formulations. Samples were analysed as a neat injection and showed no tropolone signal in any of the samples (FIG. 5).

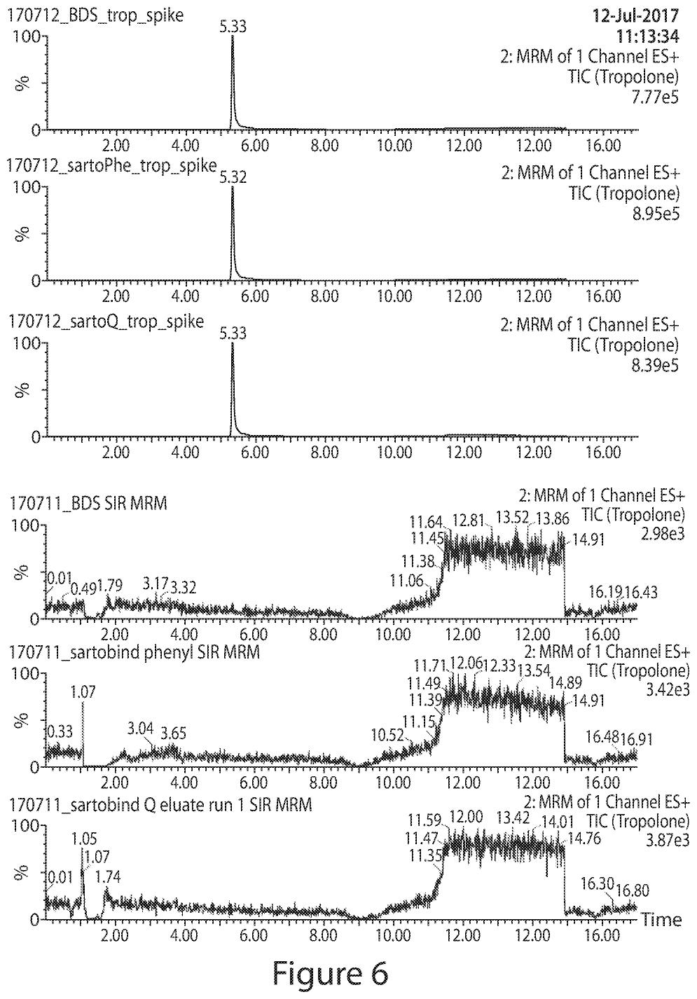

As part of method performance evaluation, tropolone standard was spiked into these in-process samples at 0.05 mg/mL (FIG. 6). This experiment confirmed the recovery of tropolone from in-process samples of various formulations. This also confirmed that tropolone was not present in the tested samples at above the lower limit of detection (LLOD) of the method (5.0 μg/mL).

### Example 5: UV Detection and the Method

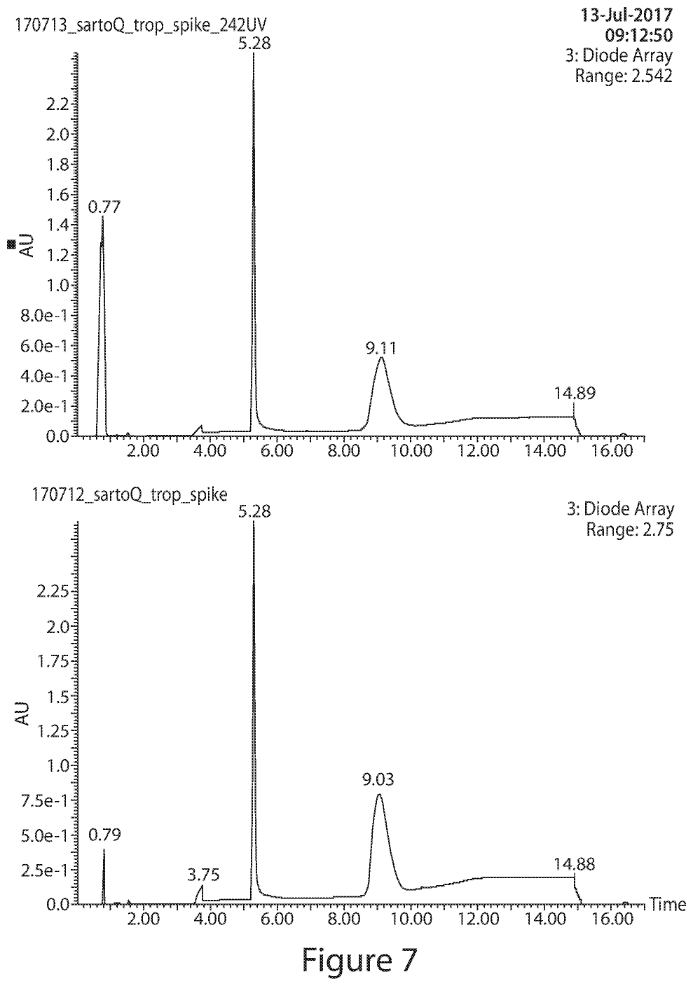

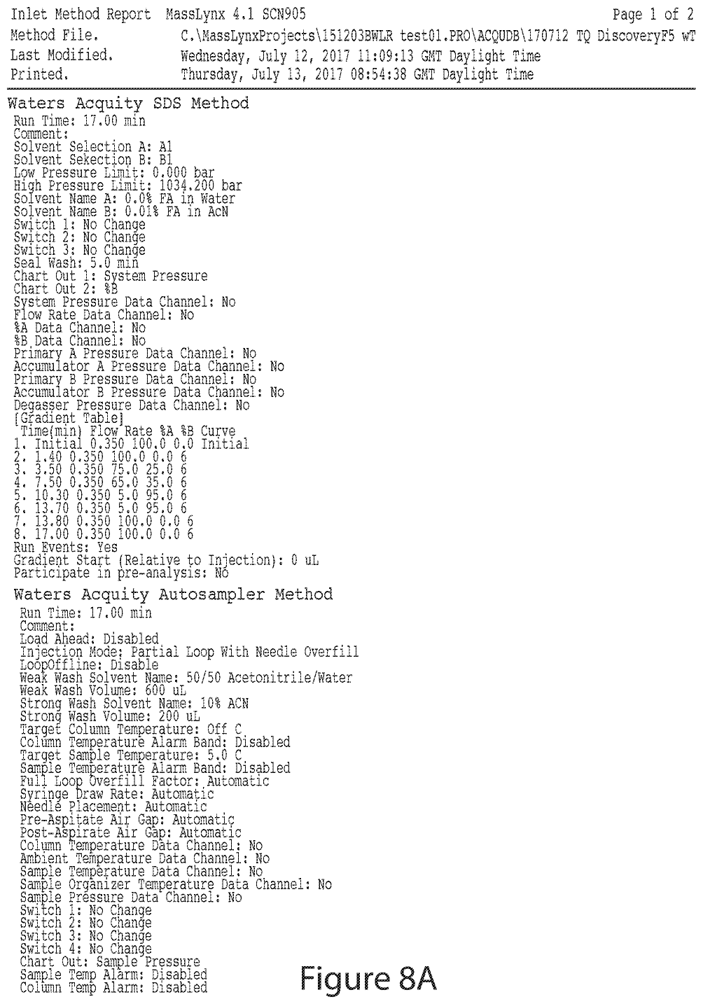

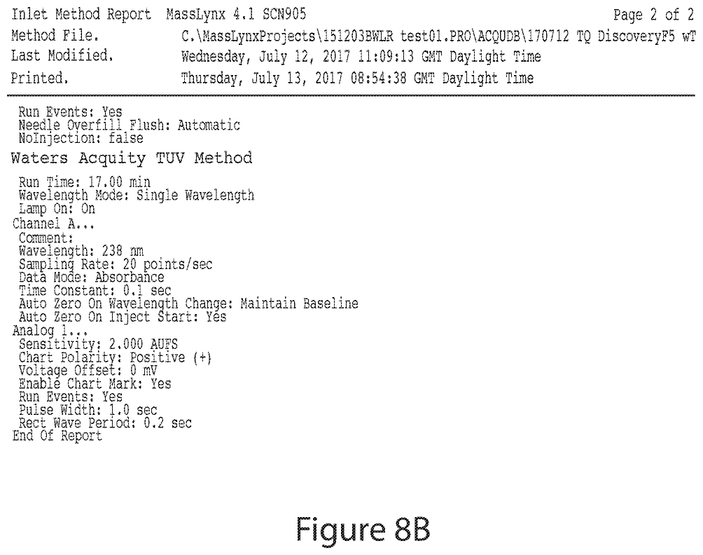

As seen in Example 2 (FIG. 1), previous methods using UV detection at 242 nm showed interfering peaks detected from the sample matrix (sample buffer peaks). It was considered whether using 238 nm absorption might increase the tropolone peak response. Using the new chromatography conditions established and tested in Examples 2-4, UV detection was tested in place of MS and the issues of interfering buffer peaks appear to have been resolved (FIG. 7). The top trace shows UV detection at 242 nm and the bottom trace at 238 nm, at both wavelengths it appears that the buffer peak previously seen at 7.0 minutes (FIG. 1) has moved retention time and no longer interferes with the tropolone peak (5.28 minutes). With increased peak resolution, MS detection may not be necessary for a new assay although it would provide greater specificity than UV detection.

### Example 6: Conclusion

A new assay for the detection of tropolone in process and purified samples was developed using LC-MS. Without optimisation this improves upon the previously used RP-HPLC-UV method by reduction of interfering peaks through altered chromatography and increased specificity of detection.

Method performance parameters were assessed for linear range, accuracy and precision with the results as follows:
Linearity: R²=0.9911
Precision=16.56% RSD
Accuracy=91.4% recovery

Using the detection wavelength of 238 nm, a small increase in tropolone peak intensity was observed when compared to 242 nm. The improved chromatographic performance obtained using the Discovery HS-F5 column and associated mobile phases resolves the issue of formulation buffer interference previously noted. It may be possible to use this LC configuration with UV detection only.

What is claimed is:

1. A method of separating a compound of Formula I, e.g., tropolone, from another component of a sample comprising:
contacting the sample with a partially or fully fluorinated alkyl or aryl, e.g., a fluorophenyl, e.g., a pentafluorophenylpropyl, moiety, under conditions wherein the compound of Formula I, e.g., tropolone, associates with, e.g., binds to or is retained by, the moiety to a greater extent than the component,
thereby separating the compound of Formula I, e.g., tropolone, from the component, wherein Formula I is: 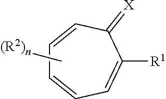
and wherein:
X is O or S;
R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;
each R² is independently C₁-C₆ alkyl, C₁-C₆ heteroalkyl, N(R^{4a})(R^{4b}), C(O)N(R^{4a})(R^{4b}), or N(R^{4a})C(O)R5; or
two R² are joined to form a heterocyclyl ring optionally substituted with one or more R⁶; or R¹ and R² are joined to form a heterocyclyl ring optionally substituted with one or more R⁶;
R³ is hydrogen, C₁-C₆ alkyl, or C₁-C₆ heteroalkyl;
R^{4a} and R^{4b} are independently hydrogen, C₁-C₆ alkyl, or C₁-C₆ heteroalkyl;
R⁵ is C₁-C₆ alkyl or C₁-C₆ heteroalkyl;
each R⁶ is independently C₁-C₆ alkyl, C₁-C₆ heteroalkyl, halo, oxo, or cyano; and
n is 0, 1, 2, 4, or 5.

2. The method of claim 1, wherein the moiety comprises a pentafluorophenylpropyl group.

3. The method of either of claims 1 or 2, wherein the pentafluorophenylpropyl group is associated with, e.g., bound to, e.g., covalently bound to, a substrate.

4. The method of claim 3, wherein the substrate comprises an insoluble substrate, e.g., a chromatography matrix, e.g., a silica gel.

5. The method of any of claims 1-4, comprising contacting the moiety with one or more mobile phases (e.g., one or two mobile phases) under conditions wherein the compound is preferentially eluted.

6. The method of any of claims 1-5, wherein the method comprises subjecting the sample to a liquid chromatography (LC) separation.

7. A method of evaluating the presence, e.g., the level, of a compound of Formula I, e.g., tropolone, in a sample comprising a product, comprising:
a) i) providing an aliquot of a sample, e.g., a compound of Formula I (e.g., tropolone) depleted phase, e.g., a mobile phase, wherein the compound of Formula I, e.g., tropolone, has been separated from another component of the sample, or
ii) subjecting the sample to conditions wherein the compound of Formula I, e.g., tropolone, is separated from another component of the sample, e.g., to form a compound of Formula I, e.g., tropolone, enriched phase or aliquot and a compound of Formula I, e.g., tropolone, depleted phase or aliquot; and
b) evaluating the presence, e.g., the level, of the compound of Formula I, e.g., tropolone, e.g., determining a value for the level of the compound of Formula I, e.g., tropolone, in the sample:
i) using tandem mass spectrometry (MS²), or
ii) using ultraviolet (UV) absorption, e.g., UV absorption at about 242 nm or about 238 nm,
thereby analyzing the sample,
wherein Formula I is: 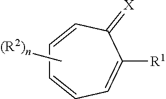
and wherein:
X is O or S;
R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;
each R² is independently C₁-C₆ alkyl, C₁-C₆ heteroalkyl, N(R^{4a})(R^{4b}), C(O)N(R^{4a})(R^{4b}), or N(R^{4a})C(O)R5; or
two R² are joined to form a heterocyclyl ring optionally substituted with one or more R⁶; or R¹ and R² are joined to form a heterocyclyl ring optionally substituted with one or more R⁶;
R³ is hydrogen, C₁-C₆ alkyl, or C₁-C₆ heteroalkyl;
R^{4a} and R^{4b} are independently hydrogen, C₁-C₆ alkyl, or C₁-C₆ heteroalkyl;
R⁵ is C₁-C₆ alkyl or C₁-C₆ heteroalkyl;
each R⁶ is independently C₁-C₆ alkyl, C₁-C₆ heteroalkyl, halo, oxo, or cyano; and
n is 0, 1, 2, 4, or 5.

8. The method of claim 7, wherein a) comprises providing an aliquot of a sample, e.g., a compound of Formula I, e.g., tropolone, depleted phase, e.g., a mobile phase, wherein the compound of Formula I, e.g., tropolone, has been separated from another component of the sample.

9. The method of claim 7, wherein a) comprises subjecting the sample to conditions wherein the compound of Formula I, e.g., tropolone, is separated from another component of the sample, e.g., to form a compound of Formula I, e.g., tropolone, enriched phase or aliquot and a compound of Formula I, e.g., tropolone, depleted phase or aliquot.

10. The method of any of any of claims 7-9, wherein a) comprises subjecting the sample to a liquid chromatography (LC) separation.

11. The method of any of claims 7-10, wherein a) comprises contacting the sample with a partially or fully fluorinated alkyl or aryl, e.g., a fluorophenyl, e.g., a pentafluorophenylpropyl, moiety, under conditions wherein the compound of Formula I, e.g., tropolone, associates with, e.g., binds to, or is retained by, the moiety to a greater extent than the component.

12. The method of claim 11, wherein the moiety comprises a pentafluorophenylpropyl group.

13. The method of any of claims 7-12, wherein b) comprises comprising evaluating the level or presence of the compound of Formula I, e.g., tropolone, e.g., determining a value for the level of the compound of Formula I, e.g., tropolone, in the sample using tandem mass spectrometry (MS²).

14. The method of any of claims 7-12, wherein b) comprises evaluating the level or presence of the compound of Formula I, e.g., tropolone, e.g., determining a value for the level of the compound of Formula I, e.g., tropolone, in the sample using ultraviolet (UV) absorption, e.g., UV absorption at about 242 nm or about 238 nm.

15. The method of any of claims 7, 11, or 12 comprising: a)i) and b)i).

16. The method of any of claims 7, 11, or 12 comprising: a)i) and b)ii).

17. The method of any of claims 7, 11, or 12 comprising: a)ii) and b)i).

18. The method of any of claims 7, 11, or 12 comprising: a)ii) and b)ii).

19. The method of any of claims 7-18, wherein the linear range of the method with regard to determining a value for the level of the compound of Formula I, e.g., tropolone, present in the sample is about 0.1-10000, 0.2-8000, 0.3-7000, 0.4-6000, 0.5-5000, 0.5-4000, 0.5-3000, 0.5-2000, or 0.5-1000 μg/ml, e.g., 0.5-1000 μg/ml.

20. The method of any of claims 7-19, wherein the lower limit of the linear range of the method with regard to determining a value for the level of the compound of Formula I, e.g., tropolone, in the sample is about 0.01, 0.05, 0.1, 0.2, 0.3, 0.35, 0.4, 0.45, 0.5, 0.6, 0.7, 0.8, 0.9, or 1 μg/ml, e.g., 0.5 μg/ml.

21. The method of any of claims 7-20, wherein the upper limit of the linear range of the method with regard to determining a value for the level of the compound of Formula I, e.g., tropolone, in the sample is about 500, 600, 700, 800, 900, 1000, 1200, 1400, 1600, 1800, 2000, 3000, 4000, 5000, 6000, 7000, 8000, 9000, or 10,000 μg/ml, e.g., 1000 μg/ml.

22. The method of any of claims 7-21, wherein the precision (e.g., represented by the standard deviation between replicate samples) of the method with regard to determining a value for the level of the compound of Formula I, e.g., tropolone, present in the sample can be less than or equal to about 50, 40, 30, 25, 20, 19, 18, 17, 16, 15, 14, 13, 12, 11, 10, 9, 8, 7, 6, 5, 4, 3, 2, or 1%, e.g., 17, 16.5, or 16%.

23. The method of any of claims 7-22, wherein the accuracy (e.g., represented by average single point spike recovery in three different samples) of the method with regard to determining a value for the level of the compound of Formula I, e.g., tropolone, present in the sample is greater than or equal to about 70, 75, 80, 81, 82, 83, 84, 85, 86, 87, 88, 89, 90, 91, 92, 93, 94, or 95%, e.g., 91%.

24. The method of any of claims 7-23, wherein the lower limit of detection of the method with regard to determining a value for the level of the compound of Formula I, e.g., tropolone, present in the sample is about 1, 1.5, 2, 2.5, 3, 3.5, 4, 4.5, 5, 5.5, 6, 6.5, 7, 7.5, 8, 8.5, 9, 9.5, or 10 μg/ml, e.g., 5 μg/ml.

25. The method of either claim 6 or 10, wherein the LC is reversed phase chromatography.

26. The method of either claim 6 or 10, wherein the LC is not reversed phase chromatography.

27. The method of either claim 6 or 10, wherein the LC comprises using a stationary phase comprising a partially or fully fluorinated alkyl or aryl, e.g., a fluorophenyl, e.g., a pentafluorophenylpropyl, group.

28. The method of claim 27, wherein the LC comprises using a stationary phase comprising a fluorophenyl group.

29. The method of claim 27, wherein the LC comprises using a stationary phase comprising a pentafluorophenylpropyl group.

30. The method of any of claims 6, 10, or 25-29, wherein the LC comprises using a first mobile phase and a second mobile phase.

31. The method of claim 30, wherein the first mobile phase comprises formic acid in water, e.g., about 0.01%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, or 1% formic acid in water.

32. The method of claim 31, wherein the first mobile phase comprises about 0.1% formic acid in water.

33. The method of claim 30, wherein the second mobile phase comprises formic acid in acetonitrile, e.g., about 0.01%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.3%, 0.4%, 0.5%, 0.6%, 0.7%, 0.8%, 0.9%, or 1% formic acid in acetonitrile.

34. The method of claim 33, wherein the second mobile phase comprises about 0.1% formic acid in acetonitrile.

35. The method of either of claims 33 or 34, wherein the second mobile phase comprises at least about 50, 55, 60, 65, 70, 75, 80, 85, 90, 95, or 100% acetonitrile, e.g., about 100% acetonitrile.

36. The method of any of claims 6, 10, or 25-35, wherein the LC comprises: using a stationary phase comprising a pentafluorophenylpropyl group, and using a first mobile phase and a second mobile phase, wherein the first mobile phase comprises about 0.1% formic acid in water, and wherein the second mobile phase comprises about 0.1% formic acid in acetonitrile.

37. The method of any of claims 6, 10, or 25-36, wherein the LC comprises using a Discovery HS F5-3 column.

38. The method of any of claims 7-13, 15, 17, and 19-37, wherein using MS² comprises selected reaction monitoring (SRM).

39. The method of any of claims 7-13, 15, 17, and 19-37, wherein using MS² comprises multiple reaction monitoring (MRM), e.g., parallel reaction monitoring (PRM).

40. The method of either of claims 38 or 39, wherein SRM or MRM (e.g., PRM), is used to monitor one or more transitions selected from transition i, ii, iii, iv, v, and vi of Table 1.

41. The method of claim 40, wherein SRM or MRM (e.g., PRM), is used to monitor transition i.

42. The method of claim 40, wherein SRM or MRM (e.g., PRM), is used to monitor transition ii.

43. The method of claim 40, wherein SRM or MRM (e.g., PRM), is used to monitor transition iii.

44. The method of claim 40, wherein SRM or MRM (e.g., PRM), is used to monitor transition iv.

45. The method of claim 40, wherein SRM or MRM (e.g., PRM), is used to monitor transition v.

46. The method of claim 40, wherein SRM or MRM (e.g., PRM), is used to monitor transition vi.

47. A reaction mixture comprising a partially or fully fluorinated alkyl or aryl, e.g., a fluorophenyl, e.g., a pentafluorophenylpropyl, moiety, and a sample comprising a compound of Formula I, e.g., tropolone, another component, and optionally a product, wherein Formula I is given by: 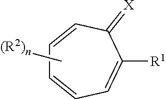
and wherein:
X is O or S;
R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;
each R² is independently C₁-C₆ alkyl, C₁-C₆ heteroalkyl, N(R^{4a})(R^{4b}), C(O)N(R^{4a})(R^{4b}), or N(R^{4a})C(O)R5; or
two R² are joined to form a heterocyclyl ring optionally substituted with one or more R⁶; or R¹ and R² are joined to form a heterocyclyl ring optionally substituted with one or more R⁶;
R³ is hydrogen, C₁-C₆ alkyl, or C₁-C₆ heteroalkyl;
R^{4a} and R^{4b} are independently hydrogen, C₁-C₆ alkyl, or C₁-C₆ heteroalkyl;
R⁵ is C₁-C₆ alkyl or C₁-C₆ heteroalkyl;
each R⁶ is independently C₁-C₆ alkyl, C₁-C₆ heteroalkyl, halo, oxo, or cyano; and
n is 0, 1, 2, 4, or 5.

48. A method of manufacturing a product, e.g., a recombinant polypeptide, comprising providing a sample comprising the product and optionally a compound of Formula I, e.g., tropolone, wherein:
the sample is analyzed by a method of any of claims 7-43, 45, or 46, or
the compound of Formula I, e.g., tropolone, is separated from another component of the sample by a method of any of claims 1-6,
wherein Formula I is given by: 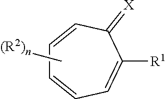
and wherein:
X is O or S;
R¹ is hydrogen, C₁-C₆ alkyl, C₁-C₆ heteroalkyl, OR³, C(O)R⁵, C(O)OR³, N(R^{4a})(R^{4b}), C(O)N(R^{4a})(R^{4b}), or N(R^{4a})C(O)R⁵;
each R² is independently C₁-C₆ alkyl, C₁-C₆ heteroalkyl, N(R^{4a})(R^{4b}), C(O)N(R^{4a})(R^{4b}), or N(R^{4a})C(O)R5; or
two R² are joined to form a heterocyclyl ring optionally substituted with one or more R⁶; or R¹ and R² are joined to form a heterocyclyl ring optionally substituted with one or more R⁶;
R³ is hydrogen, C₁-C₆ alkyl, or C₁-C₆ heteroalkyl;
R^{4a} and R^{4b} are independently hydrogen, C₁-C₆ alkyl, or C₁-C₆ heteroalkyl;
R⁵ is C₁-C₆ alkyl or C₁-C₆ heteroalkyl;
each R⁶ is independently C₁-C₆ alkyl, C₁-C₆ heteroalkyl, halo, oxo, or cyano; and
n is 0, 1, 2, 4, or 5.

49. The method of claim 48, wherein the method of manufacturing comprises expression and secretion from a plurality of cells (e.g., a plurality of CHO cell, e.g., a plurality of GS-CHO cells).

50. The method or reaction mixture of any of claims 1-49, wherein the sample comprises culture supernatant.

51. The method or reaction mixture of any of claims 1-49, wherein the sample comprises cell lysate.

52. The method or reaction mixture of any of claims 1-51, wherein the sample comprises culture supernatant and cell lysate.

53. The method or reaction mixture of any of claims 1-52, wherein the sample was generated by a method of manufacturing a product, e.g., a recombinant polypeptide.

54. The method or reaction mixture of any of claims 1-53, wherein the sample comprises a final product, e.g., a final product formulated for delivery (e.g., administration to a patient).

55. The method or reaction mixture of any of claims 1-54, wherein the product or recombinant polypeptide is a homopolymeric or heteropolymeric polypeptide, e.g., a hormone, growth factor, receptor, antibody, cytokine, receptor ligand, transcription factor or enzyme, preferably an antibody or an antibody fragment, e.g., a human antibody or a humanized antibody or fragment thereof, e.g., a humanized antibody or fragment thereof derived from a mouse, rat, rabbit, goat, sheep, or cow antibody, typically of rabbit origin.

56. The method or reaction mixture of any of claims 1-55, wherein the product or recombinant polypeptide is a therapeutic polypeptide.

57. The method or reaction mixture of any of claims 1-56, wherein the product or recombinant polypeptide is one disclosed in Table 1, Table 2, Table 3, or Table 4.

58. The method or reaction mixture of any of claims 1-57, wherein the product or recombinant polypeptide is an antibody.

59. The method or reaction mixture of claim 58, wherein the antibody is a monoclonal antibody.

60. The method or reaction mixture of either of claims 58 or 59, wherein the monoclonal antibody is a therapeutic antibody.

61. The method or reaction mixture of any of claims 49-60, wherein the cells are mammalian cells.

62. The method or reaction mixture of claim 61, wherein the cell is a mouse, rat, Chinese hamster, Syrian hamster, monkey, ape, dog, horse, ferret, or cat.

63. The method or reaction mixture of claim 61, wherein the cells are Chinese hamster ovary (CHO) cells.

64. The method or reaction mixture of claim 63, wherein the CHO cells are CHO-K1 cells, CHO-K1 SV cells, DG44 CHO cells, DUXB11 CHO cells, CHOS cells, CHO GS knock-out cells, CHO FUT8 GS knock-out cells, CHOZN cells, or CHO-derived cells.

65. The method or reaction mixture of claim 61, wherein the cells are HeIa, HEK293, HT1080, H9, HepG2, MCF7, Jurkat, NIH3T3, PC12, PER.C6, BHK (baby hamster kidney cell), VERO, SP2/0, NS0, YB2/0, Y0, EB66, C127, L cell, COS, e.g., COS1 and COS7, QC1-3, or any cells derived therefrom.
